(19) United States
(12) Patent Application Publication
(10) Pub. No.: US 2019/0226949 A1
(43) Pub. Date: Jul. 25, 2019

(54) IRIDIUM COMPLEXES FOR CELLULAR IMAGING

(71) Applicants: UNIVERSITY OF SOUTH AUSTRALIA, Adelaide (AU); CURTIN UNIVERSITY, Perth (AU)

(72) Inventors: Doug BROOKS, Cheltenham (AU); Sally PLUSH, Mile End (AU); Stefano STAGNI, Bologna (IT); Massimiliano MASSIMILIANO, Wilson (AU)

(73) Assignees: University of South Australia, Adelaid (AU); Curtin University, Perth (AU)

(21) Appl. No.: 16/318,170

(22) PCT Filed: Jul. 18, 2017
(86) PCT No.: PCT/AU2017/050740

§ 371 Date: Jan. 16, 2019

(30) Foreign Application Priority Data
Jul. 18, 2016 (AU) 2016902815

(51) Int. Cl. G01N 1/30; G01N 33/574; A61K 49/00

(52) U.S. Cl. CPC G01N 1/30; G01N 2001/302; A61K 49/006; G01N 33/57434

(57) ABSTRACT

The present disclosure relates to methods and products for imaging or labelling cells. Certain embodiments of the present disclosure provide a method of intracellular imaging of a cell. The method comprises exposing the cell to a complex comprising a phenylpyridine iridium (III) (and/or a functional derivative thereof) and a tetrazolato compound and imaging the complex in the cell.

### PRIORITY CLAIM

This application claims priority to Australian Provisional Patent Application 2016902815 filed on 18 Jul. 2016, the content of which is hereby incorporated by reference.

### FIELD

The present disclosure relates to methods and products for imaging or labelling cells.

### BACKGROUND

Molecular probes are important tools in both research and medical diagnosis. A variety of different types of molecular probes are available. Some of these probes target specific types of biological species, such as DNA, RNA or proteins, while other probes can be used to target specific cells or target specific cell structures, such as the use of antibodies that bind to cells, or antibodies or small molecules specific to proteins associated with certain cell structures.

The ability to image or label specific intracellular cell structures is particularly useful. However, the repertoire of molecular probes with this ability is limited. In addition, some probes are only suitable for cells that have been fixed or are no longer viable. The suite of probes that can be used for intracellular imaging of live cells is more limited. Probes that have the ability to be used on live cells and which also label or detect intracellular components provide a number of advantages.

Indeed, the identification of probes that are suitable for live cell imaging or labelling has become an important area for development. The ability to intracellular image or label live cells not only has important implications for visualizing normal cell function, but also has direct significance for the investigation of many diseases. Such probes also have the potential to provide diagnostic or prognostic tools that can be applied to discern specific patient pathologies.

As such, there is a need for the identification of new agents that have the ability to image or label cells, and in particular agents that have the ability to intracellularly image or label cells.

### SUMMARY

The present disclosure relates to methods and products for imaging or labelling cells.

Certain embodiments of the present disclosure provide a method of intracellular imaging of a cell, the method comprising exposing the cell to a complex comprising a phenylpyridine iridium (III) (and/or a functional derivative thereof) and a tetrazolato compound and imaging the complex in the cell.

Certain embodiments of the present disclosure provide a method of labelling a cell, the method comprising exposing the cell to a complex comprising a phenylpyridine iridium (III) (and/or a functional derivative thereof) and a tetrazolato compound and labelling the cell with the complex.

Certain embodiments of the present disclosure provide a method of labelling or detecting endoplasmic reticulum, lipid droplets and/or acidic vesicles in a cell, the method comprising exposing the cell to a neutral complex comprising a phenylpyridine iridium (III) (and/or a functional derivative thereof) and a tetrazolato compound and labelling or detecting the endoplasmic reticulum, the lipid droplets and/or the acidic vesicles in the cell.

Certain embodiments of the present disclosure provide a method of labelling or detecting mitochondria in a cell, the method comprising exposing the cell to a cationic complex comprising a phenylpyridine iridium (III) (and/or a functional derivative thereof) and a tetrazolato compound and labelling or detecting the mitochondria in the cell.

Certain embodiments of the present disclosure provide a method of identifying a cancerous cell, the method comprising exposing the cell to a complex comprising a phenylpyridine iridium (III) (and/or a functional derivative thereof) and a tetrazolato compound, and identifying the cell as a cancerous cell on the basis of altered labelling of the cell with the complex and/or altered localisation of the complex in the cell.

Certain embodiments of the present disclosure provide a method of identifying a cancerous prostate cell, the method comprising exposing a cell to a complex comprising a phenylpyridine iridium (III) (and/or a functional derivative thereof) and a tetrazolato compound, and identifying the cell as a cancerous prostate cell on the basis of altered labelling of the cell with the complex and/or altered localisation of the complex in the cell.

Certain embodiments of the present disclosure provide a method of screening for a cancerous cell, the method comprising exposing a cell to a complex comprising a phenylpyridine iridium (III) (and/or a functional derivative thereof) and a tetrazolato compound, and identifying the cell as a cancerous cell on the basis of altered labelling of the cell with the complex and/or altered localisation of the complex in the cell.

Certain embodiments of the present disclosure provide a method of screening for a cancerous prostate cell, the method comprising exposing a cell to a complex comprising a phenylpyridine iridium (III) (and/or a functional derivative thereof) and a tetrazolato compound, and identifying the cell as a cancerous prostate cell on the basis of altered labelling of the cell with the complex and/or altered localisation of the complex in the cell.

Certain embodiments of the present disclosure provide a method of identifying a prostate cancer in a subject, the method comprising exposing a cell from the subject to a complex comprising a phenylpyridine iridium (III) (and/or a functional derivative thereof) and a tetrazolato compound and identifying prostate cancer in the subject on the basis of altered labelling of the cell with the complex and/or altered localisation of the complex in the cell.

Certain embodiments of the present disclosure provide a kit for labelling cells, the kit comprising a complex comprising a phenylpyridine iridium (III) (and/or a functional derivative thereof) and a tetrazolato compound.

Certain embodiments of the present disclosure provide a kit for intracellular imaging of cells, the kit comprising a complex comprising a phenylpyridine iridium (III) (and/or a functional derivative thereof) and a tetrazolato compound.

Certain embodiments of the present disclosure provide a cell labelling agent, the agent comprising a complex comprising a phenylpyridine iridium (III) (and/or a functional derivative thereof) and a tetrazolato compound.

Certain embodiments of the present disclosure provide an intracellular imaging agent, the agent comprising a complex comprising a phenylpyridine iridium (III) (and/or a functional derivative thereof) and a tetrazolato compound.

Certain embodiments of the present disclosure provide a composition for labelling a cell, the composition comprising a complex comprising a phenylpyridine iridium (III) (and/or a functional derivative thereof) and a tetrazolato compound.

Certain embodiments of the present disclosure provide a composition for intracellular imaging of a cell, the composition comprising a complex comprising a phenylpyridine iridium (III) (and/or a functional derivative thereof) and a tetrazolato compound.

Certain embodiments of the present disclosure provide a method of identifying a complex for intracellular imaging or labelling of a cell, the method comprising:
providing a candidate complex comprising a complex comprising a phenylpyridine iridium (III) (and/or a functional derivative thereof) and a tetrazolato compound;
determining the ability of the candidate complex to intracellularly image or label a cell; and
identifying the candidate complex as a complex for intracellular imaging or labelling of a cell.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows neutral and cationic iridium tetrazolato complexes synthesized and investigated in this work.

FIG. 2 shows a summary of the synthetic procedures performed in this work.

FIG. 3 shows X-ray crystal structures of the neutral [Ir(F₂ppy)₂(TzPyCN)](left) and the cationic [Ir(ppy)₂(MeTzPyPhCN)]⁺ (right), with displacement ellipsoinds at 50% probability. Lattice solvent molecules and counter ion are omitted.

FIG. 4 shows absorption spectra of the various complexes from diluted (10⁻⁵M) dichloromethane solutions. Panel A shows spectra for [Ir(ppy)₂(TzPyCN], [Ir(F₂ppy)₂(TzPyCN], and [Ir(ppy)₂(TzPyPhCN]. Panel B shows spectra for [Ir(ppy)₂(MeTzPyCN]⁺, [Ir(F₂ppy)₂(MeTzPyCN]⁺, and [Ir(ppy)₂(MeTzPyPhCN]⁺. Panel C shows spectra for [Ir(ppy)₂(TzQn] and [Ir(ppy)₂(TziQn]. Panel D shows spectra for [Ir(ppy)₂(MeTzQn]⁺ and [Ir(ppy)₂(MeTziQn]⁺.

FIG. 5 shows emission spectra of the complexes from diluted (10⁻⁵ M) dichloromethane solutions. Panel A shows spectra for [Ir(ppy)₂(TzPyCN], [Ir(F₂ppy)₂(TzPyCN], and [Ir(ppy)₂(TzPyPhCN]. Panel B shows spectra for [Ir(ppy)₂(MeTzPyCN]⁺, [Ir(F₂ppy)₂(MeTzPyCN]⁺, and [Ir(ppy)₂(MeTzPyPhCN]⁺. Panel C shows spectra for [Ir(ppy)₂(TzQn] and [Ir(ppy)₂(TziQn]. Panel D shows spectra for [Ir(ppy)₂(MeTzQn]⁺ and [Ir(ppy)₂(MeTziQn]⁺.

FIG. 6 shows staining of live H9c2 rat cardiomyoblast cells with neutral iridium complexes (left panel) [Ir(ppy)₂(TzPyCN)], [Ir(F₂ppy)₂(TzPyCN)], and [Ir(ppy)₂(TzPyPhCN)], incubated for 30 minutes at 20 μM. Co-staining of the same cells with ER-Tracker™ Red and BODIPY® 500/510 C₁, C₁₂ (middle panel). Merged images (right panel).

FIG. 7 shows staining of live H9c2 rat cardiomyoblast cells with neutral iridium complexes (left panel) [Ir(ppy)₂(TzQn)] and [Ir(ppy)₂(TziQn)], incubated for 30 minutes at 20 μM. Co-staining of the same cells with ER-Tracker™ Red and BODIPY® 500/510 C₁, C₁₂ (middle panel). Merged images (right panel).

FIG. 8 shows staining of live H9c2 rat cardiomyoblast cells with cationic methylated iridium complexes (left panel), incubated for 30 minutes at 20 μM. Co-staining of the same cells with ER-Tracker™ Red, MitoTracker® Red CMXRos, and β-BODIPY® FL C₅-HPC (middle panel). Merged images (right panel).

FIG. 9 shows cell viability after 24 hours incubation with the iridium complexes at 20 (Panel A) and 40 μM (Panel B). Neutral complexes are represented by dark grey bars, whereas cationic complexes are represented by mid-grey green bars. 0) Control (light grey bars); 1) [Ir(ppy)₂(TzPyCN)]; 2) [Ir(ppy)₂(MeTzPyCN)]⁺; 3) [Ir(F₂ppy)₂(TzPyCN)]; 4) [Ir(F₂ppy)₂(MeTzPyCN)]⁺; 5) [Ir(ppy)₂(TzPyPhCN)]; 6) [Ir(ppy)₂(MeTzPyPhCN)]⁺; 7) [Ir(ppy)₂(TzQn)]; 8) [Ir(ppy)₂(MeTzQn)]⁺; 9) [Ir(ppy)₂(TziQn)]; 10) [Ir(ppy)₂(MeTziQn)]⁺.

FIG. 10 shows atom labelling for NMR spectra. The labelling is consistent for the corresponding neutral complexes and non-methylated tetrazolato ligands.

FIG. 11 shows prostate IraZolve [Ir(ppy)₂(TzPyCN)] distribution (green, a′, b′) in LNCaP prostate cancer cells in relation to the (a) endoplasmic reticulum detected by ER-Tracker® (red, a″) or (b) to fatty acids accumulated in lipid droplets detected by BODIPY 500/510 (red, b″). Scale bar 20 um.

FIG. 12 shows distribution of altered lipids in prostate cancer cells. Panels (a-p) shows micrographs of cross-section through the prostate cells showing intracellular localisation of neutral and polar lipids. The cholesterol was depicted by staining cells with Filipin (a-d). The neutral lipids, such as triglycerides and cholesteryl esters, were detected by staining cells with BODIPY® 493/503 (e-h). ReZolve-L1™ (i-l) and IraZolve [Ir(ppy)₂(TzPyCN)] (m-p) were used for staining polar lipids and lipid droplets, respectively. Representative images were from (a, e, i, m) non-malignant control PNT1a and prostate cancer (b, f, j, n) DU145, (c, g, k, o) 22RV1 and (d, h, l, p) LNCaP cell lines. Visualised prostate cells were (a-h) fixed in paraformaldehyde and (i-p) live. Scale bar, 20 μm.

### DETAILED DESCRIPTION

The present disclosure relates to methods and products for imaging or labelling cells.

The present disclosure is based on the recognition that certain iridium complexes have specificity for binding to certain intracellular structures. The complexes also have a variety of useful properties, including for example one or more of a small molecular size, they are adaptable to chemical modification, they exhibit emissive properties that are attributable to the core iridium ion, they unexpectedly have the ability to stain live cells and therefore may be used as live cell imaging agents, and they also may be used to enable the visualization of pathogenic processes.

Certain embodiments of the present disclosure provide a method of imaging of a cell.

Certain embodiments of the present disclosure provide a method of intracellular imaging of a cell, the method comprising exposing the cell to a complex comprising a phenylpyridine iridium (III) (and/or a functional derivative thereof) and a tetrazolato compound, and imaging the complex in the cell.

In certain embodiments, the phenylpyridine iridium complex comprises a functional derivative thereof. For example, the phenylpyridine iridium complex may be a substituted derivative thereof, such as a substitute derivative of a phenylpyridine, and/or a substituted derivative of the tetrazolato compound.

The ability of complexes to intracellularly image cells (or eg tissues with cells) may be determined by a method as described herein. In this regard, the term “image” or variants thereof as used herein refers to the ability of an agent to visualise, observe, label, detect, stain, or otherwise allow identification of a target.

In certain embodiments, the phenylpyridine iridium complex comprises a biphenylpyridine iridium complex.

In certain embodiments, the phenylpyridine iridium complex comprises a non-substituted phenylpyridine in the complex. In certain embodiments, the phenylpyridine iridium complex comprises a substituted phenylpyridine in the complex. For example, the phenylpyridine in the complex may be a fluoro-substituted derivative, such as a monofluoro-substituted or a difluoro-substituted derivative.

In certain embodiments, the phenylpyridine iridium complex comprises a substituted phenylpyridine. In certain embodiments, the phenylpyridine iridium complex comprises a fluorine-substituted phenylpyridine. Other substituted derivatives are contemplated.

In certain embodiments, the tetrazolato compound comprises a nitrogen containing aromatic heterocyclic group. Other groups are contemplated.

In certain embodiments, the nitrogen containing aromatic heterocyclic group comprises a pyridyl or a pyrazinyl group and/or a functional derivative thereof. In certain embodiments, the pyridyl group or the pyrazinyl group is a substituted group. For example, the pyridyl group or the pyrazinyl group may comprise a cyano-substituted group. Other derivatives are contemplated.

In certain embodiments, the tetrazolato compound comprises a functional derivative of a tetrazolato group, such as a substituted tetrazolato group.

In certain embodiments, the tetrazolato group comprises a substituted tetrazolato group. In certain embodiments, the tetrazolato group comprises an alkyl substituted tetrazolato group. In certain embodiments, the tetrazolato group comprises a methyl-substituted tetrazolato group.

In certain embodiments, the tetrazolato group comprises a 3-substituted tetrazolato group.

In certain embodiments, the tetrazolato group comprises a 3-methyl substituted tetrazolato group.

Methods for producing complexes as described herein are known, for example as described in Stagni et al. (2008) Inorg. Chem 47: 10509-10521 and Fiorini et al. (2016) Dalton Trans. 45: 3256-3259. Methods for testing the ability of a complex to intracellularly image or label a cell are described herein.

In certain embodiments, the complex comprises one or more of the following complexes:

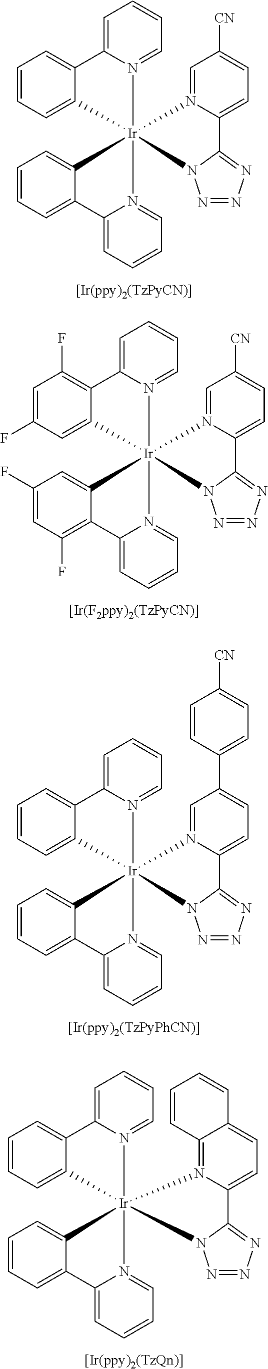

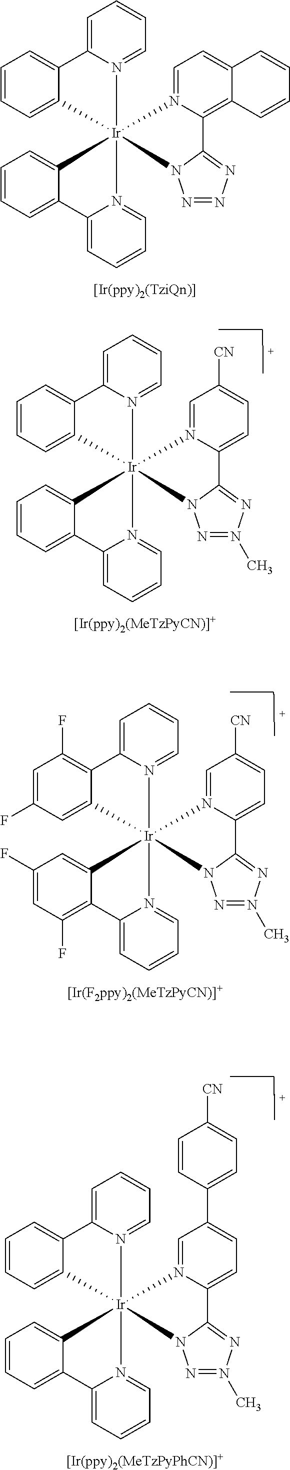

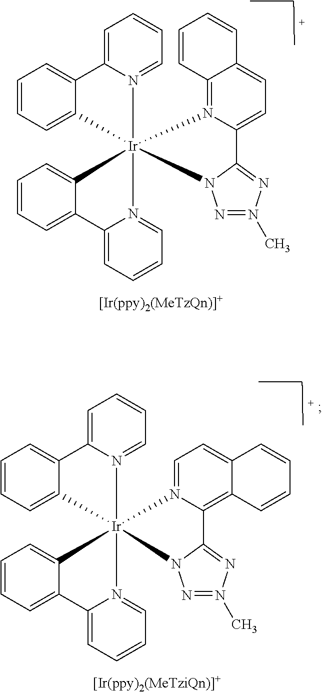

and/or an isomer thereof, a substituted derivative thereof, or a salt thereof. Isomers include structural isomers, stereoisomers, geometric isomers, and optical isomers.

Methods for imaging complexes are as described herein, and include in vitro imaging and in vivo imaging. For example, a complex as described herein may be used to image a subject, such as one or more tissues or regions of a subject.

In certain embodiments, the complex is a neutral complex. In certain embodiments, the neutral complex comprises one or more of [Ir(ppy)₂(TzPyCN], [Ir(F₂ppy)₂(TzPyCN], [Ir(ppy)₂(TzPyPhCN], [Ir(ppy)₂(TzQn] and [Ir(ppy)₂(TziQn].

In certain embodiments, the complex is a cationic complex. In certain embodiments, the cationic complex comprises one or more of [Ir(F₂ppy)₂(MeTzPyCN]⁺, [Ir(ppy)₂(MeTzPyPhCN]⁺, [Ir(ppy)₂(MeTzQn]⁺ and [Ir(ppy)₂(MeTziQn]⁺.

In certain embodiments, the complex is the complex alone. In certain embodiments, the complex comprises another group, moiety, or agent associated with or conjugated to the complex.

In certain embodiments, the cell is a live cell, a dead cell, a fixed cell, a cell in vivo, an ex vivo cell, a cell in a tissue or region, and/or a cell in a biological fluid. In certain embodiments, the cell is a cell in vitro. The term “cell” as used herein refers to one or more cells.

In certain embodiments, the cell is present in vivo, in a cell sample, a sample of live cells, a tissue, a tissue sample, a cell extract, a biopsy, a bodily fluid sample, a blood sample, a urine sample, a saliva sample, flow cytometric sample, a biological sample, and/or an extract, component, derivative, processed form or purified form of any of the aforementioned. For example, the cell may be a circulating tumour cell in a blood sample.

The term “exposing”, and related terms such as “expose” and “exposure” as used herein, refers to contacting and/or treating a species with an effective amount of a complex as described herein. The term includes for example exposing a cell in vitro to a complex as described herein, exposing a cell in vivo to a complex as described herein, and administering a complex as described herein to a subject so as to label cells in vivo.

In certain embodiments, the method comprises exposing the cell to one complex. In certain embodiments, the method comprises exposing the cell to one or more complexes. In certain embodiments, the method comprises exposing the cell to two complexes or three complexes. In certain embodiments, the method comprises exposing the cell to multiple complexes.

Method for exposing species to a complex, including administration of agents to a subject, are known in the art.

Examples of subjects include humans and animals, such as livestock animals (eg a horse, a cow, a sheep, a goat, a pig), a domestic animal (eg a dog or a cat) and other types of animals such as monkeys, rabbits, mice and laboratory animals, and insects. Veterinary applications of the present disclosure are contemplated. Use of any of the aforementioned animals as animal models for use is also contemplated.

In certain embodiments, the cell is in a biological sample. Methods for obtaining biological samples are known in the art, and include biopsies, tissue samples, and biological fluid samples, such as blood samples. In certain embodiments, the method comprises obtaining cells from a subject. Methods for obtaining samples are known in the art.

In certain embodiments, the biological sample comprises a cell in vivo, an ex vivo cell and/or a cell in a biological fluid. In certain embodiments, the biological sample comprises a cell in vitro. In certain embodiments, the biological sample comprises a flow cytometric cell.

Examples of biological samples include a cell sample, a sample of live cells, a cell extract, a cell lysate, a cell-free sample, a sorted cell, a non-fixed cell, a fixed cell, a biopsy, a tissue sample, a bodily fluid sample, a blood sample, a urine sample, a saliva sample, a tissue section, mounted cells, a tissue sample, a flow cytometric sample, a drug doping sample and/or an extract, component, derivative, processed form or purified form of any of the aforementioned.

In certain embodiments, the complexes are substantially non-cytotoxic.

In certain embodiments, the cell is a live cell and the complex is substantially non-cytotoxic to the cell. Methods for determining cytotoxicity are known in the art.

In certain embodiments, the cell is a fixed cell. Methods for fixing cells are known in the art.

In certain embodiments, the method is used to image a cellular structure in a cell. In certain embodiments, the method comprises imaging a cellular structure in a cell. In certain embodiments, the complex localises to or labels a cellular structure. In certain embodiments, the complex localises to or labels an intracellular structure.

In certain embodiments, the cellular structure comprises an intracellular structure. In certain embodiments, the cellular structure comprises one or more of an endoplasmic reticulum, acid vesicles, small intracellular vesicles, lipid droplets and mitochondria. Other types of cellular structures are contemplated.

In certain embodiments, the cellular structure comprises one or more lipids. In certain embodiments, the cellular structure is a lipid containing structure.

In certain embodiments, the cellular structure is a structure in a live cell, a dead cell, a non-fixed cell, a sorted cell, a fixed cell, a stained cell, an in vivo cell, an ex vivo cell, an in vitro cell, or a cell of a specific type or associated with a pathology, such as a cancer cell. Other types of cells are described herein.

In certain embodiments, the complex is a neutral complex and the complex localises to/labels one or more of endoplasmic reticulum, lipid droplets, small intracellular vesicles and acidic vesicles in the cell.

In certain embodiments, the neutral complex comprises one or more of [Ir(ppy)₂(TzPyCN], [Ir(F₂ppy)₂(TzPyCN], [Ir(ppy)₂(TzPyPhCN], [Ir(ppy)₂(TzQn] and [Ir(ppy)₂(TziQn] and the complex localises to/labels one or more of endoplasmic reticulum, lipid droplets, small intracellular vesicles and acidic vesicles in the cell.

In certain embodiments, the complex is a cationic complex and the complex localises to/labels mitochondria in the cell.

In certain embodiments, the cationic complex comprises one or more of [Ir(F₂ppy)₂(MeTzPyCN]⁺, [Ir(ppy)₂(MeTzPyPhCN]⁺, [Ir(ppy)₂(MeTzQn]⁺ and [Ir(ppy)₂(MeTziQn]⁺ and the complex localises to/labels mitochondria in the cell.

In certain embodiments, the complex is an internalised complex.

In certain embodiments, the complex shows differential imaging/labelling to different cells. For example, the complex may show altered binding or localisation to different cells.

In certain embodiments, the complex shows differential labelling to a cancer cell. For example, the complex may show altered binding or localisation to a cancer cell.

In certain embodiments, the complex shows increased labelling in a cancerous cell as compared to a non-cancerous cell. In certain embodiments, the complex shows increased labelling of the endoplasmic reticulum and small intracellular vesicles in a cancerous cell as compared to a non-cancerous cell.

In certain embodiments, the cancer cell is a prostate cancer cell or a breast cancer cell or other cancer cell. In this regard, the complexes as described herein may be used to distinguish cancerous cells from non-cancerous cells and as such may be used for diagnostic or prognostic purposes. For example, circulating tumour cells in blood may be detected using the complexes described herein, such as by detecting circulating tumour cells from a patient in a blood sample, such as by flow cytometry.

In certain embodiments, the method is used for imaging of a live cell, in vivo imaging, to detect or label a cellular structure, to detect cells, to detect or label a non-cancerous cell and/or a cancerous cell, to identify a non-cancerous cell or a cancerous cell, to screen for cancerous cells, to distinguish a cancerous cell from a non-cancerous cell, to detect cancer in a subject, to identify cancer in a subject, to detect circulating cancerous cells, to screen for cancer in a subject, for diagnosis of cancer in a subject, and/or for prognosis of cancer in a subject.

Certain embodiments of the present disclosure provide a method of labelling a cell.

Certain embodiments of the present disclosure provide a method of labelling a cell, the method comprising exposing the cell to a complex comprising a phenylpyridine iridium (III) (and/or a functional derivative thereof) and a tetrazolato compound and labelling the cell with the complex.

Complexes comprising a phenylpyridine iridium (III) (and/or a functional derivative thereof) and a tetrazolato compound are as described herein.

Cells are as described herein. Methods for exposing complexes to a cell(s) to label the cell(s) are as described herein.

In certain embodiments, the complex is a neutral complex. Neutral complexes are as described herein.

In certain embodiments, the complex is a neutral complex and the method comprises labelling or detecting endoplasmic reticulum, lipid droplets and/or acidic vesicles in the cell.

In certain embodiments, the complex is a cationic complex. Cationic complexes are as described herein.

In certain embodiments, the complex is a cationic complex and the method comprises labelling or detecting mitochondria in the cell.

In certain embodiments, the method is used to label or detect a cellular structure in a cell. Cellular structures are as described herein.

Certain embodiments of the present disclosure provide a method of labelling or detecting a cellular structure in a cell, the method comprising exposing the cell to a complex comprising a phenylpyridine iridium (III) (and/or a functional derivative thereof) and a tetrazolato compound and labelling or detecting the cellular structure in the cell with the complex.

Certain embodiments of the present disclosure provide a method of labelling or detecting endoplasmic reticulum, lipid droplets, small intracellular vesicles and/or acidic vesicles in a cell, the method comprising exposing the cell to a neutral complex comprising a phenylpyridine iridium (III) (and/or a functional derivative thereof) and a tetrazolato compound and labelling or detecting the endoplasmic reticulum, the lipid droplets, small intracellular vesicles and/or the acidic vesicles in the cell.

Certain embodiments of the present disclosure provide a method of labelling or detecting mitochondria in a cell, the method comprising exposing the cell to a cationic complex comprising a phenylpyridine iridium (III) (and/or a functional derivative thereof) and a tetrazolato compound and labelling or detecting the mitochondria in the cell.

Certain embodiments of the present disclosure provide a method of identifying or detecting a cancerous cell. For example, such methods may be used to screen for cancer in a subject.

Certain embodiments of the present disclosure provide a method of identifying a cancerous cell, the method comprising exposing the cell to a complex comprising a phenylpyridine iridium (III) (and/or a functional derivative thereof) and a tetrazolato compound, and identifying the cell as a cancerous cell on the basis of altered labelling of the cell with the complex and/or altered localisation of the complex in the cell.

In certain embodiments, the altered labelling comprises increased labelling in a cancerous cell as compared to a non-cancerous cell. In certain embodiments, the altered labelling comprises increased labelling of the endoplasmic reticulum and small intracellular vesicles in a cancerous cell as compared to a non-cancerous cell.

In certain embodiments, the cancerous cell is a prostate cancer cell or a breast or other cancer cell.

Certain embodiments of the present disclosure provide a method of identifying a cancerous prostate cell or a cancerous breast cell, the method comprising exposing the cell to a complex comprising a phenylpyridine iridium (III) (and/or a functional derivative thereof) and a tetrazolato compound, and identifying the cell as a cancerous prostate cell or a cancerous breast cell on the basis of altered labelling of the cell with the complex and/or altered localisation of the complex in the cell.

Certain embodiments of the present disclosure provide a method of screening for a cancerous cell.

Certain embodiments of the present disclosure provide a method of screening for a cancerous cell, the method comprising exposing a cell to a complex comprising a phenylpyridine iridium (III) (and/or a functional derivative thereof) and a tetrazolato compound, and identifying the cell as a cancerous cell on the basis of altered labelling of the cell with the complex and/or altered localisation of the complex in the cell.

In certain embodiments, the altered labelling comprises increased labelling in a cancerous cell as compared to a non-cancerous cell. In certain embodiments, the altered labelling comprises increased labelling of the endoplasmic reticulum, lipid droplets and/or small intracellular vesicles in a cancerous cell as compared to a non-cancerous cell.

In certain embodiments, the cancerous cell is a prostate cancer cell or a breast cancer cell.

Certain embodiments of the present disclosure provide a method of screening for a cancerous prostate cell or a cancerous breast cell, the method comprising exposing a cell to a complex comprising a phenylpyridine iridium (III) (and/or a functional derivative thereof) and a tetrazolato compound, and identifying the cell as a cancerous prostate cell or a cancerous breast cell on the basis of altered labelling of the cell with the complex and/or altered localisation of the complex in the cell.

In certain embodiments, the altered labelling comprises increased labelling in a cancerous cell as compared to a non-cancerous cell.

In certain embodiments, the altered labelling comprises increased labelling/staining of the endoplasmic reticulum, lipid droplets and/or small intracellular vesicles in a cancerous cell as compared to a non-cancerous cell.

In certain embodiments, low, barely detectable or reduced labelling may be indicative of a non-cancerous cell. In certain embodiments, low, barely detectable or reduced labelling of the endoplasmic reticulum, lipid droplets and/or small intracellular vesicles may be indicative of a non-cancerous cell.

In certain embodiments, increased labelling may be indicative of a cancerous cell. In certain embodiments, increased labelling of the endoplasmic reticulum, lipid droplets and/or small intracellular vesicles may be indicative of a cancerous cell.

In certain embodiments, the method comprises obtaining cells from a subject for screening. Methods for obtaining samples are known in the art.

In certain embodiments, the method is used to identify a cancer in the subject. In certain embodiments, the method is used to exclude the presence of cancer in the subject. In certain embodiments, the method is used to screen for a cancer in the subject.

Certain embodiments of the present disclosure provide a method of identifying a cancer in a subject, using a method as described herein.

Certain embodiments of the present disclosure provide a method of identifying a cancer in a subject, the method comprising exposing a cell from the subject to a complex comprising a phenylpyridine iridium (III) (and/or a functional derivative thereof) and a tetrazolato compound and identifying cancer in the subject on the basis of altered labelling of the cell with the complex and/or altered localisation of the complex in the cell.

In certain embodiments, the method comprises obtaining cells from the subject for labelling to identify cancer in the subject. In certain embodiments, the method comprises exposing a cell in vivo in the subject to identify cancer in the subject.

In certain embodiments, the cancer is a prostate cancer or a breast cancer.

Certain embodiments of the present disclosure provide a method of identifying a prostate cancer in a subject, the method comprising exposing a cell from the subject to a complex comprising a phenylpyridine iridium (III) (and/or a functional derivative thereof) and a tetrazolato compound and identifying prostate cancer in the subject on the basis of altered labelling of the cell with the complex and/or altered localisation of the complex in the cell.

Certain embodiments of the present disclosure provide a method of detecting a cancer in a subject.

Certain embodiments of the present disclosure provide a method of detecting a cancer in a subject, the method comprising exposing a cell from the subject to a complex comprising a phenylpyridine iridium (III) (and/or a functional derivative thereof) and a tetrazolato compound and identifying cancer in the subject on the basis of altered labelling of the cell with the complex and/or altered localisation of the complex in the cell.

In certain embodiments, the method comprises obtaining cells from the subject for labelling to identify/detect cancer in the subject. For example, the method may be used to detect circulating tumour cells from a patient in a blood sample by flow cytometry, or detecting cancerous cells in a tissue sample or biopsy. In certain embodiments, the method comprises exposing a cell in vivo in the subject to detect cancer in the subject.

In certain embodiments, the cancer is a prostate cancer or a breast cancer or other cancer.

Certain embodiments of the present disclosure provide a method of detecting a prostate cancer in a subject, the method comprising exposing a cell from the subject to a complex comprising a phenylpyridine iridium (III) (and/or a functional derivative thereof) and a tetrazolato compound and detecting prostate cancer in the subject on the basis of altered labelling of the cell with the complex and/or altered localisation of the complex in the cell.

Certain embodiments of the present disclosure provide a kit for performing a method as described herein.

Certain embodiments of the present disclosure provide a kit for labelling cells, the kit comprising a complex comprising a phenylpyridine iridium (III) (and/or a functional derivative thereof) and a tetrazolato compound.

Certain embodiments of the present disclosure provide a kit for intracellular imaging of cells, the kit comprising a complex comprising a phenylpyridine iridium (III) (and/or a functional derivative thereof) and a tetrazolato compound.

In certain embodiments, a kit as described herein further comprises instructions and/or reagents.

For example, the kit may also include instructions and/or reagents for using the complex, instructions and/or reagents for exposing the cells to the complex, and/or instructions and/or reagents for imaging the cells, including enhancers, stabilisers and controls.

Certain embodiments of the present disclosure provide a cell labelling agent comprising a complex as described herein.

Certain embodiments of the present disclosure provide a cell labelling agent, the agent comprising a complex comprising a phenylpyridine iridium (III) (and/or a functional derivative thereof) and a tetrazolato compound.

In certain embodiments, the cell labelling agent comprises the complex alone. In certain embodiments, the complex comprises another group, moiety, or agent associated with or conjugated to the complex.

Certain embodiments of the present disclosure provide an intracellular imaging agent comprising a complex as described herein.

Certain embodiments of the present disclosure provide an intracellular imaging agent, the agent comprising a complex comprising a phenylpyridine iridium (III) (and/or a functional derivative thereof) and a tetrazolato compound.

In certain embodiments, the intracellular imaging agent comprises the complex alone. In certain embodiments, the complex comprises another group, moiety, or agent associated with or conjugated to the complex.

Certain embodiments of the present disclosure provide a composition for labelling a cell, the composition comprising a complex as described herein.

In certain embodiments, the complex comprises another group, moiety, or agent associated with or conjugated to the complex.

Certain embodiments of the present disclosure provide a composition for labelling a cell, the composition comprising a complex comprising a phenylpyridine iridium (III) (and/or a functional derivative thereof) and a tetrazolato compound.

Certain embodiments of the present disclosure provide a composition for intracellular imaging of a cell, the composition comprising a complex comprising a phenylpyridine iridium (III) (and/or a functional derivative thereof) and a tetrazolato compound.

Certain embodiments of the present disclosure provide a method for labelling a cell, the method comprising using an agent, or a composition as described herein.

Certain embodiments of the present disclosure provide a method of imaging a cell, the method comprising using an agent or a composition as described herein.

Certain embodiments of the present disclosure provide a method of identifying a complex for intracellular imaging or labelling of a cell. Such methods may be used to screen new reagents for research and diagnostic/prognostic purposes.

Certain embodiments of the present disclosure provide a method of identifying a complex for intracellular imaging or labelling of a cell, the method comprising:
providing a candidate complex comprising a complex comprising a phenylpyridine iridium (III) (and/or a functional derivative thereof) and a tetrazolato compound;
determining the ability of the candidate complex to intracellularly image or label a cell; and
identifying the candidate complex as a complex for intracellular imaging or labelling of a cell.

Complexes comprising a phenylpyridine iridium (III) (and/or a functional derivative thereof) and a tetrazolato compound are as described herein.

In certain embodiments, the candidate complex comprises a derivative of one of the following complexes:

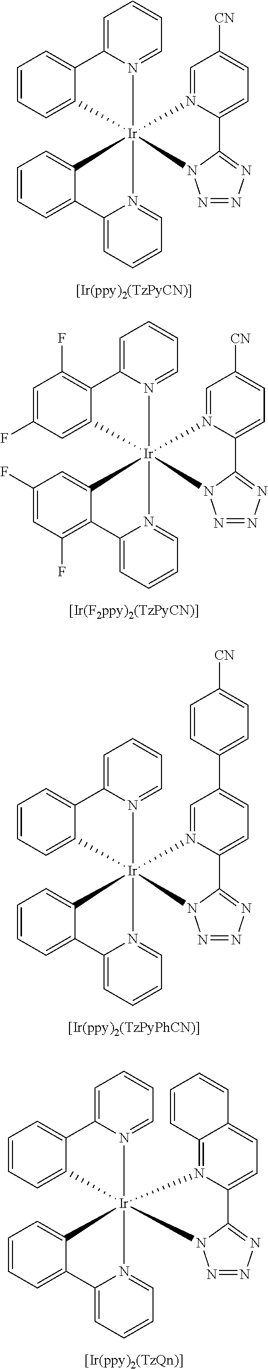

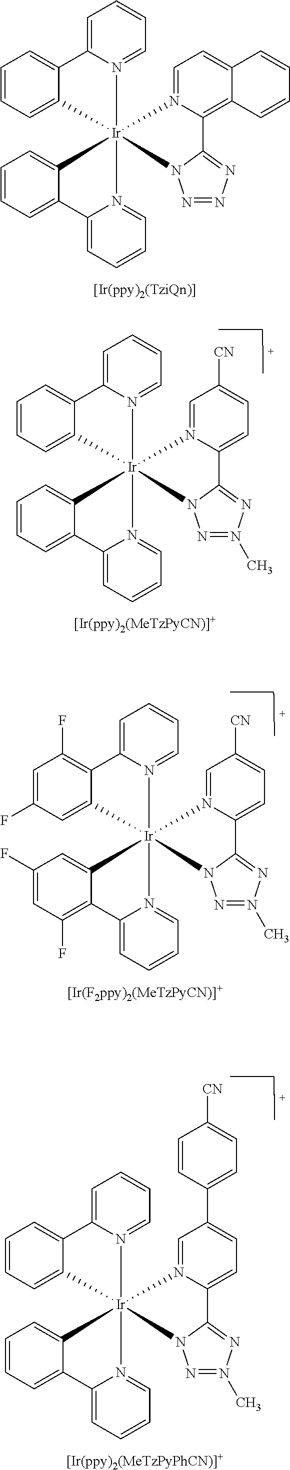

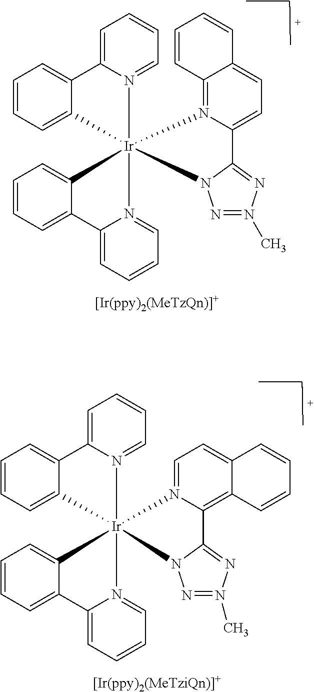

In certain embodiments, the derivative of the compound comprises a substituted derivative, a derivative replacing a functional group or another group, or a moiety or agent associated with or conjugated to the complex. In certain embodiments, the derivative comprises an isomer of the compound. Isomers are as described herein.

Certain embodiments of the present disclosure provide a complex identified by the method.

Certain embodiments of the present disclosure provide use of a complex as described herein, for example to label or detect a cellular structure as described herein, or detect or label cells as described herein.

Certain embodiments of the present disclosure provide use of a complex as described herein to detect a disease, condition or state, such as a cancer.

Certain embodiments of the present disclosure provide a method of detecting a disease, condition or state in a subject using a complex as described herein. For example, changes in biology associated with some intracellular structures may be indicative of certain pathological processes.

Certain embodiments of the present disclosure provide use of a complex as described herein to identify a subject suffering from, or susceptible to, a disease, condition or state.

Certain embodiments of the present disclosure provide use of a complex as described herein for diagnosis and/or prognosis.

Certain embodiments of the present disclosure provide a method of identifying a subject suffering from, or susceptible to, a disease, condition or state using a complex as described herein.

Certain embodiments of the present disclosure provide a complex as described herein.

Certain embodiments of the present disclosure provide a complex comprising a phenylpyridine iridium (III) (and/or a functional derivative thereof) and a tetrazolato compound.

Complexes comprising a phenylpyridine iridium (III) and a tetrazolato compound are as described herein. Methods for synthesizing such complexes are as described herein.

Certain embodiments of the present disclosure provide a complex selected from one of the following complexes:

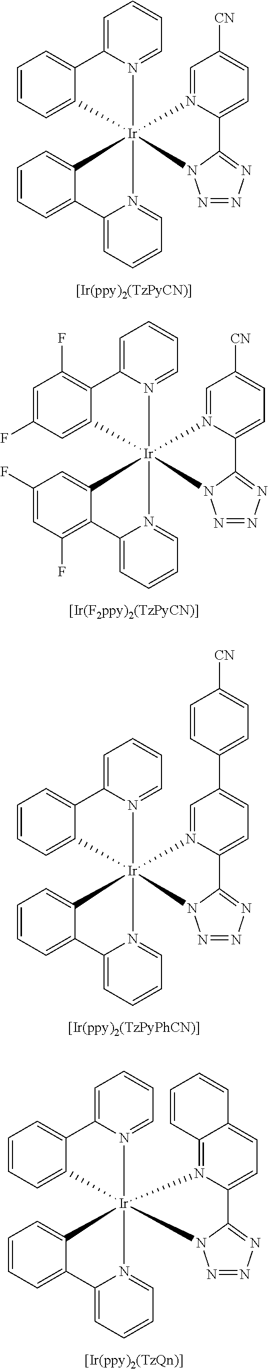

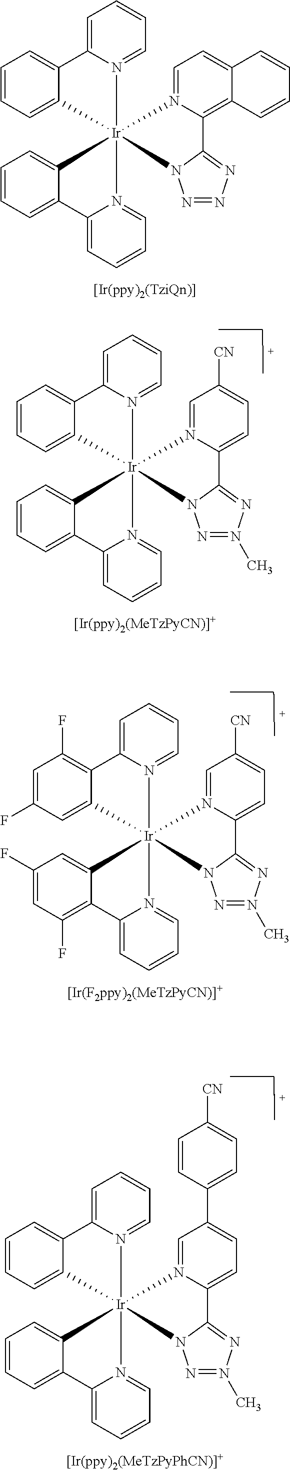

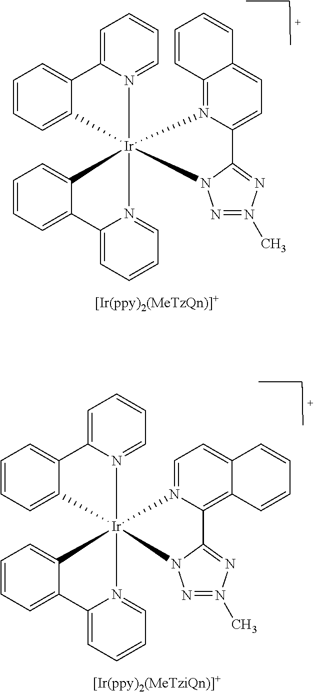

and/or a salt, a derivative, or an isomer of any of the aforementioned complexes.

The present disclosure is further described by the following examples. It is to be understood that the following description is for the purpose of describing particular embodiments only and is not intended to be limiting with respect to the above description.

### Example 1—Iridium Tetrazolato Complexes as Cellular Labels

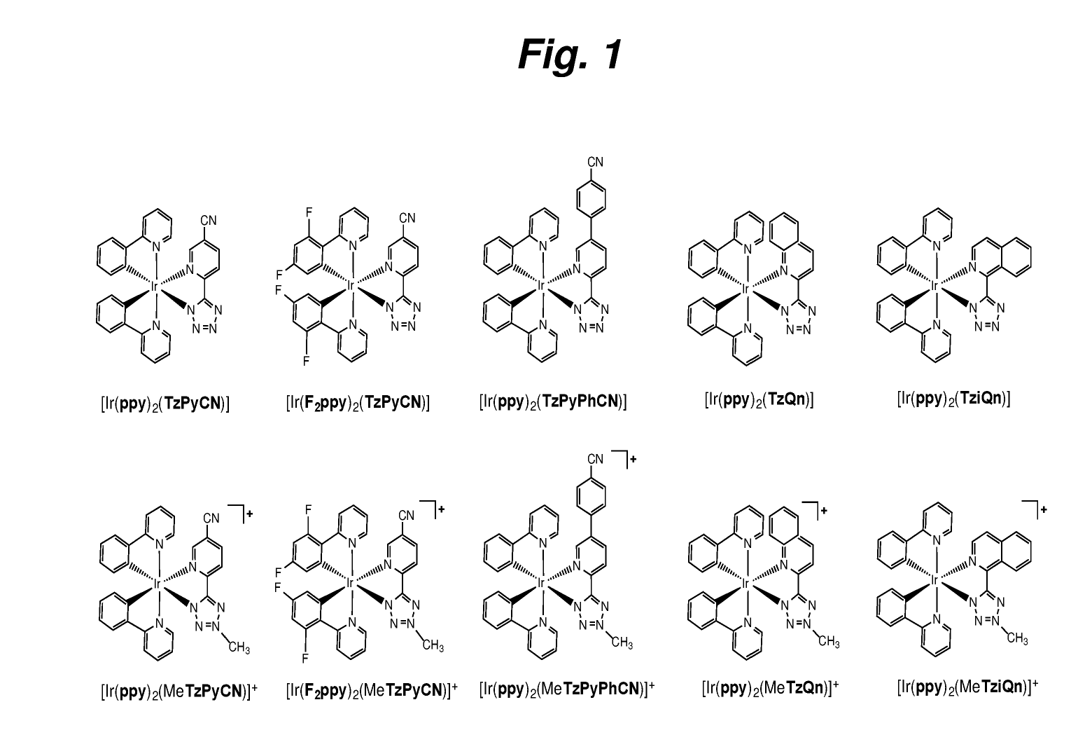

Neutral and cationic iridium tetrazolato complexes synthesized and investigated in this work are shown in FIG. 1.

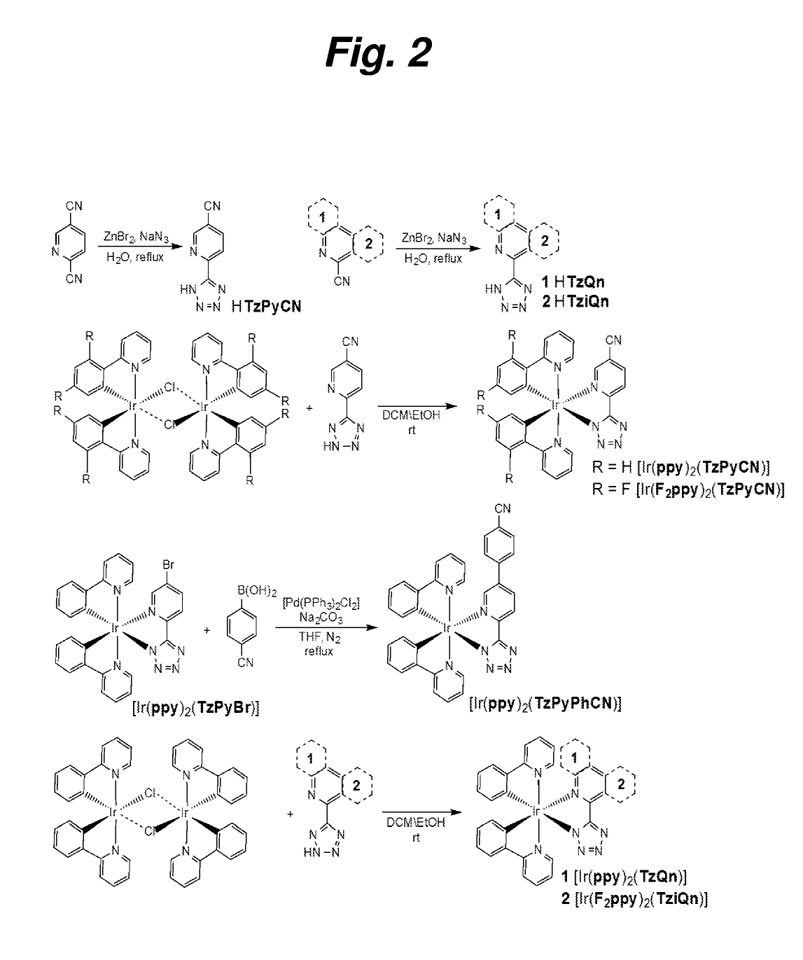

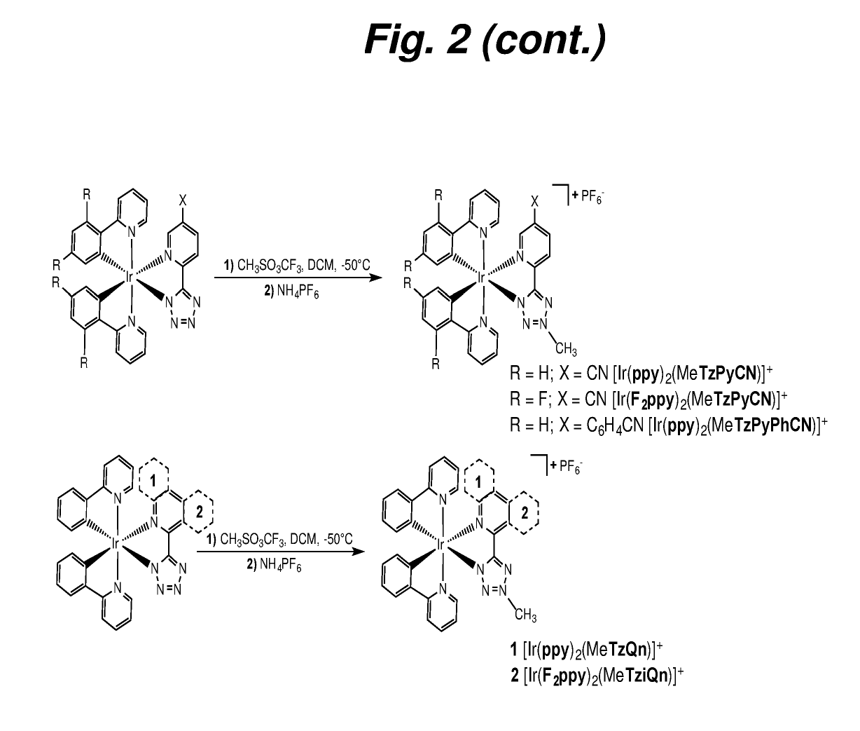

The ligand precursor 2,5-dicyanopyridine was obtained in three steps starting from commercially available 2,5-pyridinedicarboxilic acid, following a Fisher esterification to form the corresponding dimethyl ester, aminolysis with aqueous ammonia to yield the corresponding primary diamide, and lastly a dehydration reaction to convert the primary amide groups to nitrile moieties. Following a methodology developed by Sharpless (FIG. 2), 2,5-dicyanopyridine was reacted with sodium azide in water at reflux and in the presence of zinc bromide, yielding the targeted HTzPyCN ligand in good yields (64%). Complete purification of HTzPyCN from the ditetrazolised by-product by selective protonation was challenging, however complete purification could be easily performed after the formation of the corresponding iridium complexes. The ligands HTzQn and HTziQn were synthesised following the same tetrazolisation procedure.

To access the family of neutral iridium complexes [Ir(ppy)₂(TzPyCN)], [Ir(F₂ppy)₂(TzPyCN)], [Ir(ppy)₂(TzQn)], and [Ir(ppy)₂(TziQn)], the corresponding chloro-bridged iridium dimer, bearing either ppy or F₂ppy as cyclometalating ligands, were reacted with a slight excess of the corresponding pyridyl or (iso)quinolyl tetrazolato species (FIG. 2), according to previously reported procedures for the preparation of analogous iridium tetrazolato complexes.

The complex [Ir(ppy)₂(TzPyPhCN)] was prepared via a different route, by Suzuki coupling of the previously reported [Ir(ppy)₂(TzPyBr)] with 4-cyanophenylboronic acid (FIG. 2). Previous attempt to react the iridium chloro-bridged dimer with the pre-formed corresponding tetrazolato ligand were unsuccessful, as an inseparable complex mixture of products was obtained.

Methylation of all the neutral complexes was performed using the same methodology, consisting of treatment of the starting material with methyl trifluoromethanesulphonate followed by metathesis with ammonium hexafluorophosphate (FIG. 2).

All the synthesised complexes were characterised by means of ¹H/¹³C NMR and IR spectroscopy as well as elemental analysis, with data consistent with their proposed formulations.

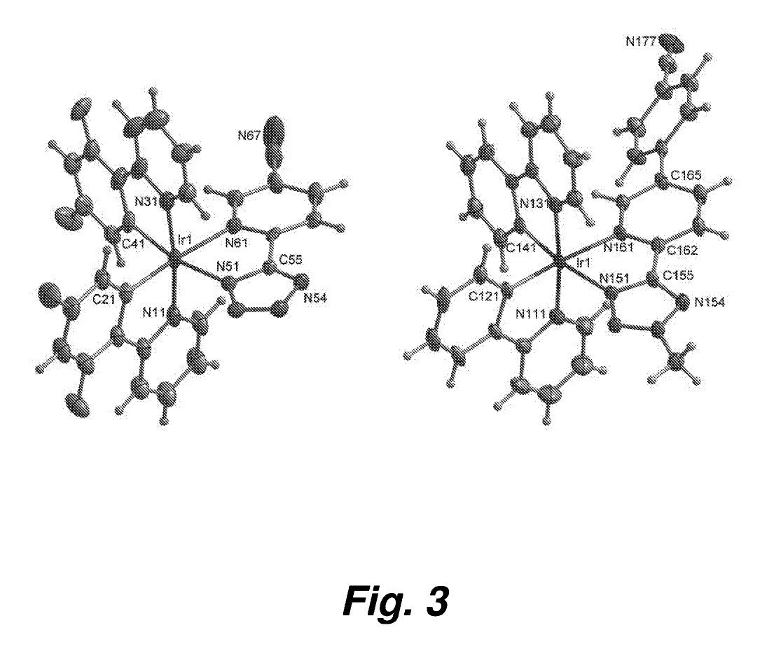

Despite several attempts, we were able to obtain single crystals suitable for X-ray diffraction only for two complexes: [Ir(F₂ppy)₂(TzPyCN)] and [Ir(ppy)₂(MeTzPyPhCN)][PF₆]. The complexes crystallise in the monoclinic I2/a and P2/n space groups, respectively. The coordination arrangement of the ligands is analogous, with the two pyridine rings of the phenylpyridine species adopting a trans configuration, while the two cyclometallated phenyl rings are arranged in cis configuration. The rest of the coordination sphere is in both cases occupied by the two nitrogen atoms of the tetrazole and pyridine rings. The [Ir(ppy)₂(MeTzPyPhCN)][PF₆] complex highlight methylation of the tetrazole ring at the N3 position, consistently with previous reports on analogous complexes (FIG. 3).

Photophysical Properties

A summary of the photophysical data for the complexes as diluted solutions (10⁻⁵ M) in dichloromethane, water, PBS, lysosomal fluid solution, and ethyl laurate is reported in Table 1. All the listed aqueous media also contain 0.1% DMSO to favour the solubilisation of the complexes. The various solvents have been chosen to identify potential modulations on the photophysical behaviour of the iridium complexes in diverse cellular environments such as cytoplasm, endosome/lysosomes acidic organelles, or lipid-rich compartments like membranes, endoplasmic reticulum, and lipid droplets. In general, the photophysical characteristics were measured in aerated solutions to take into account the presence of oxygen in live cells. On the other hand, dichloromethane solutions were compared as aerated and degassed to confirm oxygen sensitivity and triplet character of the excited states of the complexes.

TABLE 1
Summary of photophysical data for the synthesised iridium complexes in
diluted (10⁻⁵M) dichloromethane and aqueous solutions.

| | Absorption | | | Emission - 298K | | | |
|---|---|---|---|---|---|---|---|
| | | λ_{abs} [nm] | λₑₘ | τₐₑᵣ/τ_{deaer} | | kᵣ | kₙᵣ |
| Complex | Solvent^{a} | (10⁴ε [M⁻¹cm⁻¹]) | [nm] | [ns]^{b} | Φ_{aer/}Φ_{deaer}^{c} | [10⁵s⁻¹]^{d} | [10⁶s⁻¹]^{e} |
| [Ir(ppy)₂(TzPyCN)] | DCM | 263 (6.57), 339 (1.59), | 618 | 131/529 | 0.055/0.397 | 7.505 | 1.140 |
| | | 382 (1.09), 420 (0.58) | | | | | |
| | H₂O | 253 (3.52), 377 (0.69) | 618 | 123 (25) | 0.047 | — | — |
| | | | | 440 (75) | | | |
| [Ir(F₂ppy)₂(TzPyCN)] | DCM | 256 (5.20), 364 (0.95) | 542 | 140/697 | 0.042/0.406 | 5.825 | 0.852 |
| | H₂O | 256 (4.83), 302 (3.36), | 552 | 189 (41) | 0.057 | — | — |
| | | 366 (1.16) | | 628 (59) | | | |
| [Ir(ppy)₂(TzPyPhCN)] | DCM | 268 (3.95), 290 (3.64), | 560 | 114/880 | 0.023/0.149 | 1.693 | 0.967 |
| | | 345 (0.58), 423 (0.27) | | | | | |
| | H₂O | 286 (1.87), 350 (0.86), | 580 | 200 (31) | 0.055 | — | — |
| | | 421 (0.34) | | 728 (69) | | | |
| [Ir(ppy)₂(MeTzPyCN)]⁺ | DCM | 266 (8.93), 376 (1.44) | 680 | 31 (79), 168 (21)/ | 0.012/0.014 | 4.000* | 28.171* |
| | | | | 35 (61)*, 362 | | 0.387** | 2.724** |
| | | | | (39)** | | | |
| | H₂O | 260 (4.44), 373 (0.59) | — | — | — | — | — |
| [Ir(F₂ppy)₂(MeTzPyCN)]⁺ | DCM | 250 (3.56), 316 (1.28), | 600 | 287/551 | 0.057/0.188 | 2.142 | 1.601 |
| | | 356 (0.58) | | | | | |
| | H₂O | 260 (4.12), 309 (1.55), | 636 | 11 (76) | 0.005 | — | — |
| | | 353 (0.66) | | 56 (24) | | | |
| [Ir(ppy)₂(MeTzPyPhCN)]⁺ | DCM | 267 (3.39), 290 (3.14), | 600 | 184/434 | 0.057/0.093 | 2.143 | 2.090 |
| | | 378 (0.40) | | | | | |
| | H₂O | 264 (3.82), 390 (0.35) | 605 | 17 (22) | 0.005 | — | — |
| | | | | 320 (88) | | | |
| [Ir(ppy)₂(TzQn)] | DCM | 263 (8.44), 345 (1.97), | 580 | 153/831 | 0.021/0.065 | 0.782 | 1.125 |
| | | 428 (0.42) | | | | | |
| | H₂O | 266 (4.41), 350 (2.35), | 600 | 305 (25) | 0.039 | — | — |
| | | 425 (0.97) | | 928 (75) | | | |
| [Ir(ppy)₂(TziQn)] | DCM | 260 (9.77), 340 (3.13), | 535, | 497/6713 | 0.036/0.144 | 0.214 | 0.128 |
| | | 424 (0.75) | 575, 620 | | | | |
| | H₂O | 268 (3.42), 350 (2.00), | 590 | 217 (33) | 0.018 | — | — |
| | | 435 (0.80) | | 531 (67) | | | |
| [Ir(ppy)₂(MeTzQn)]⁺ | DCM | 257 (9.29), 376 (0.95) | 635 | 218/546 | 0.028/0.043 | 0.788 | 1.753 |
| | H₂O | 251 (8.30), 307 (2.52), | 630 | 17 (39) | 0.004 | — | — |
| | | 364 (1.09) | | 134 (61) | | | |
| [Ir(ppy)₂(MeTziQn)]⁺ | DCM | 253 (6.96), 303 (2.45), | 640 | 208/485 | 0.049/0.063 | 1.299 | 1.932 |
| | | 371 (1.24) | | | | | |
| | H₂O | 251 (6.64), 291 (2.60), | 660 | 9 (53) | 0.004 | — | — |
| | | 355 (1.36) | | 93 (47) | | | |

^{a}Aqueous solutions contain 0.1% of DMSO.
^{b}For the biexponential excited state lifetime (τ), the relative weights of the exponential curves are reported in parentheses.
^{c}Measured versus Ru(bpy)₃²⁺ in water (φᵣ = 0.028).
^{d}Calculated as [φ/τ] using measurements from deaerated solutions.
^{e}Calculated as [(1 − φ)/τ] using measurements from deaerated solutions.

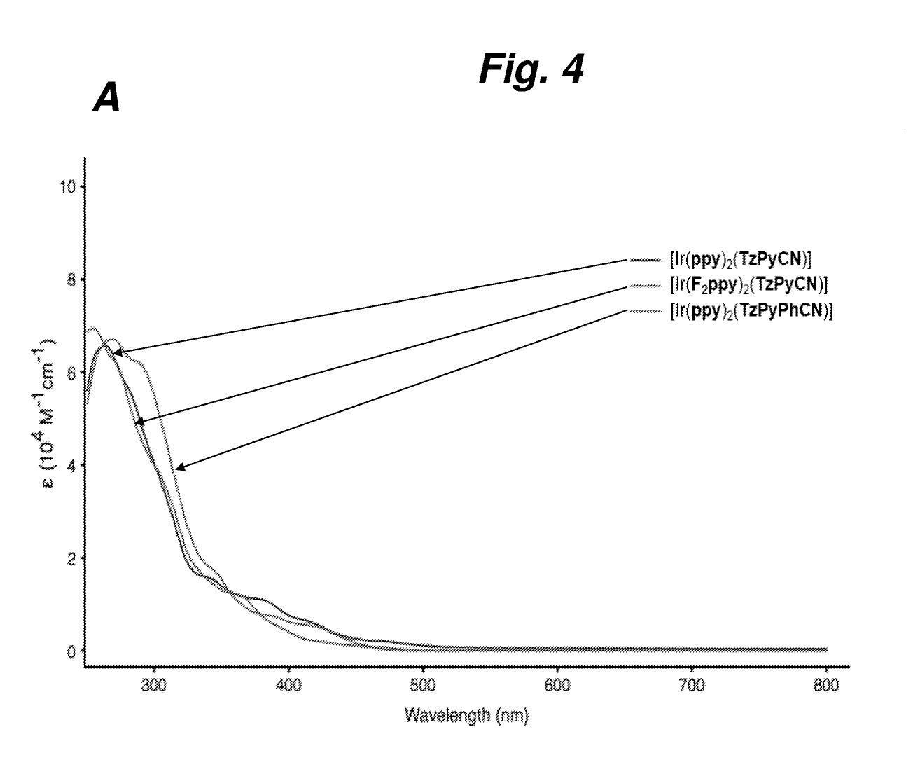

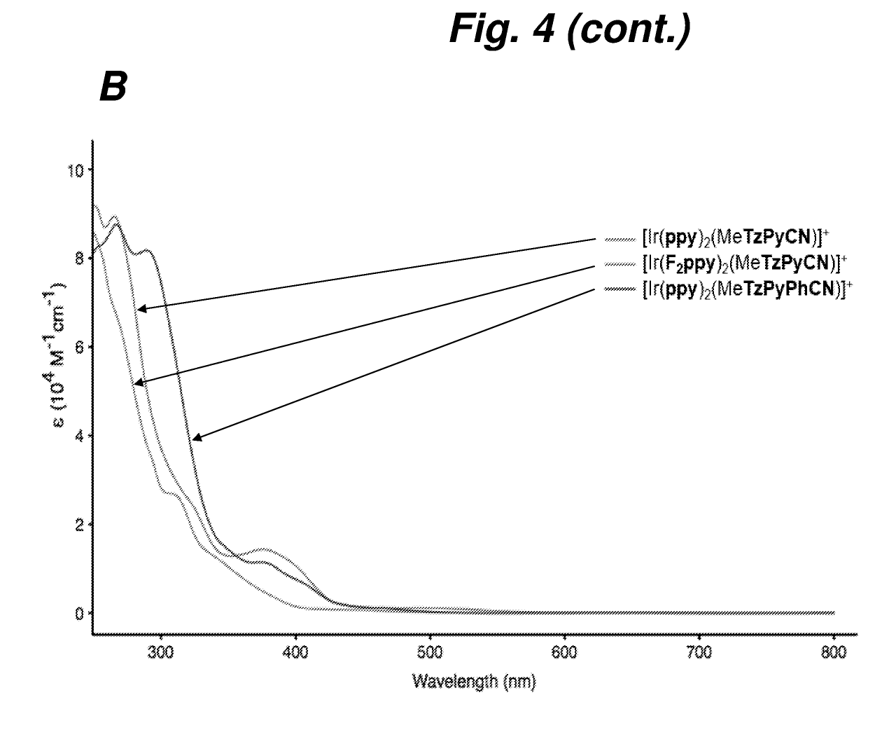

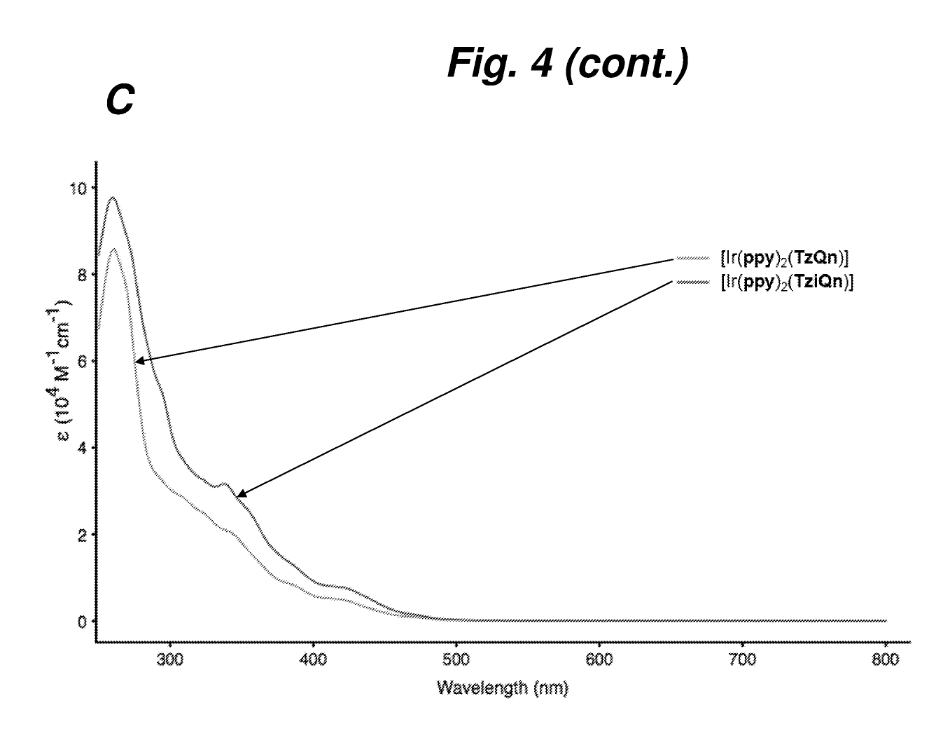

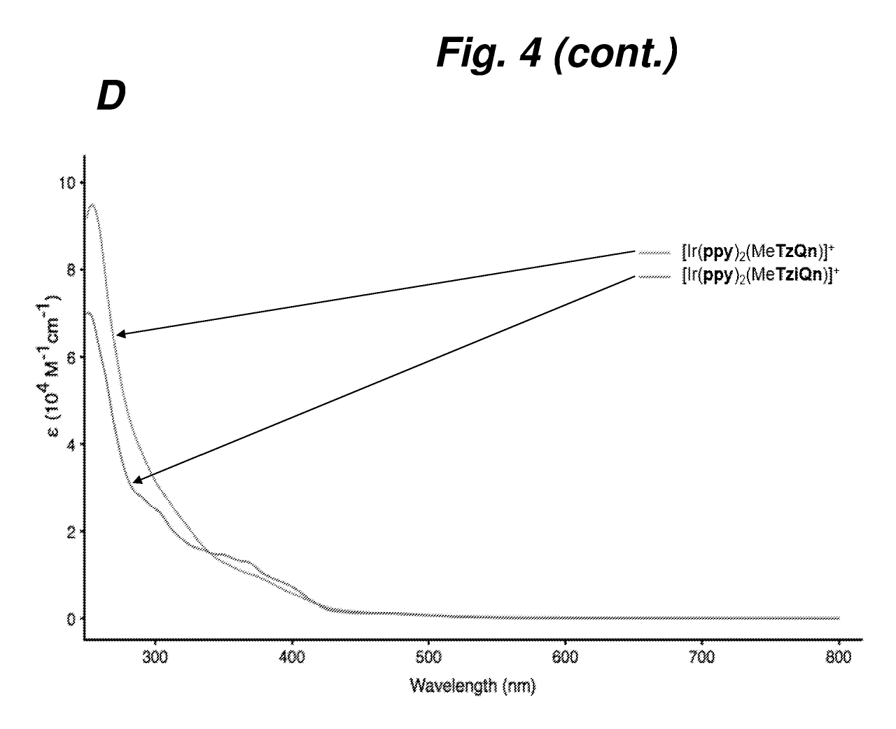

The absorption spectra of the complexes in diluted dichloromethane solutions are reported in FIG. 4. The spectra present analogous appearance and are characterised by intense absorption bands in the region 250-300 nm, which are associated with ligand-centered (LC) π-π* transitions, followed by bands of lower intensity tailing off into the visible region of the spectrum. These lower energy bands are associated to admixtures of spin allowed (singlet to singlet) and spin forbidden (singlet to triplet) ligand-to-ligand (LLCT) and metal-to-ligand charge transfer (MLCT) transitions, as typical for previously studied iridium tetrazolato complexes.

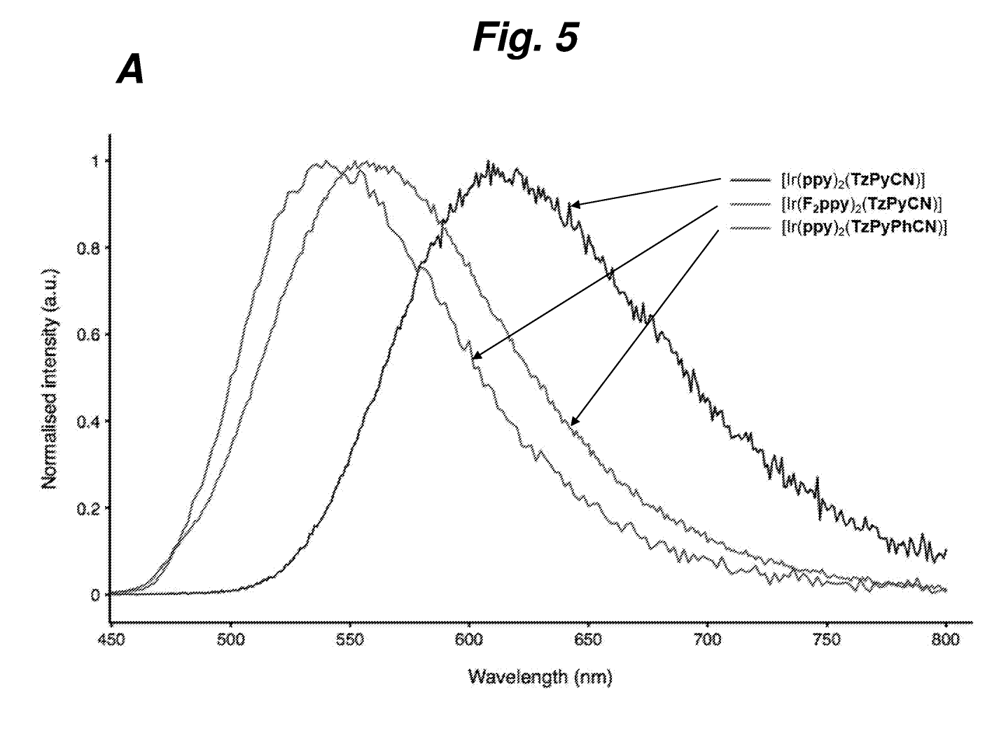

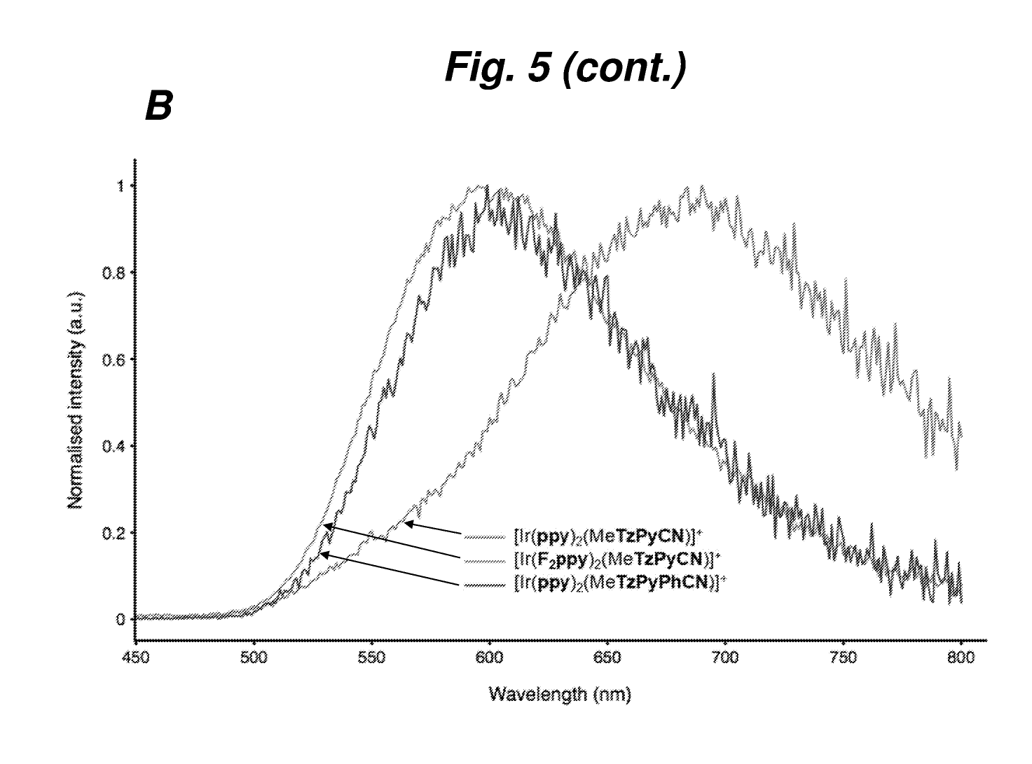

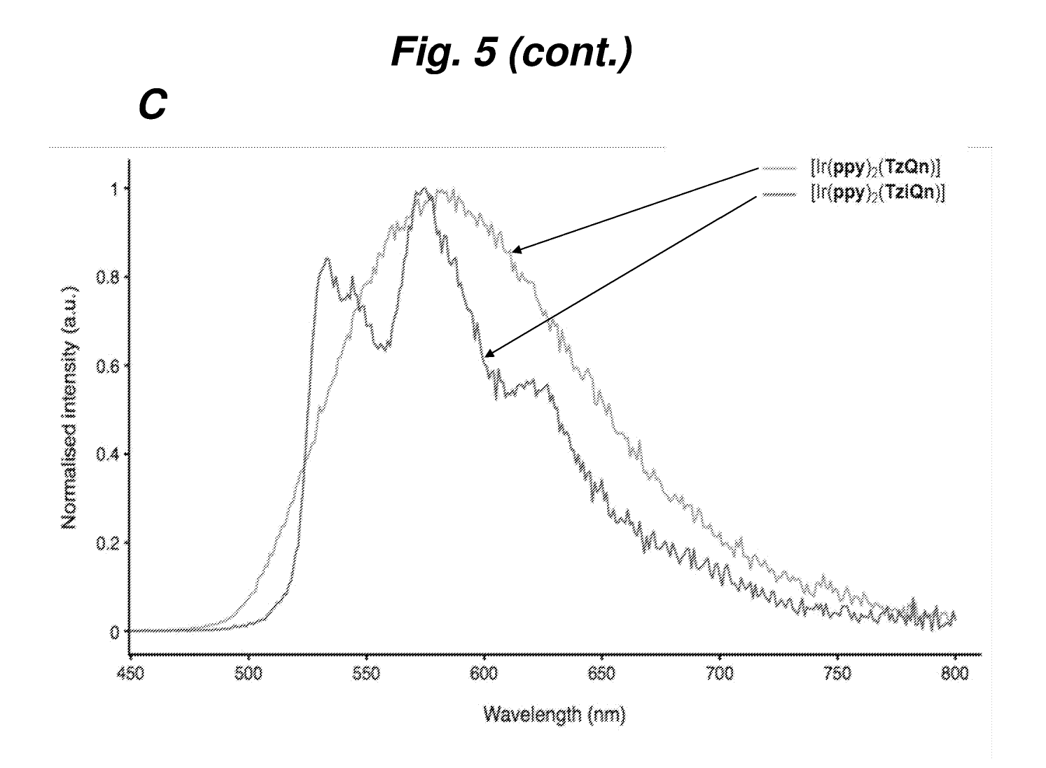

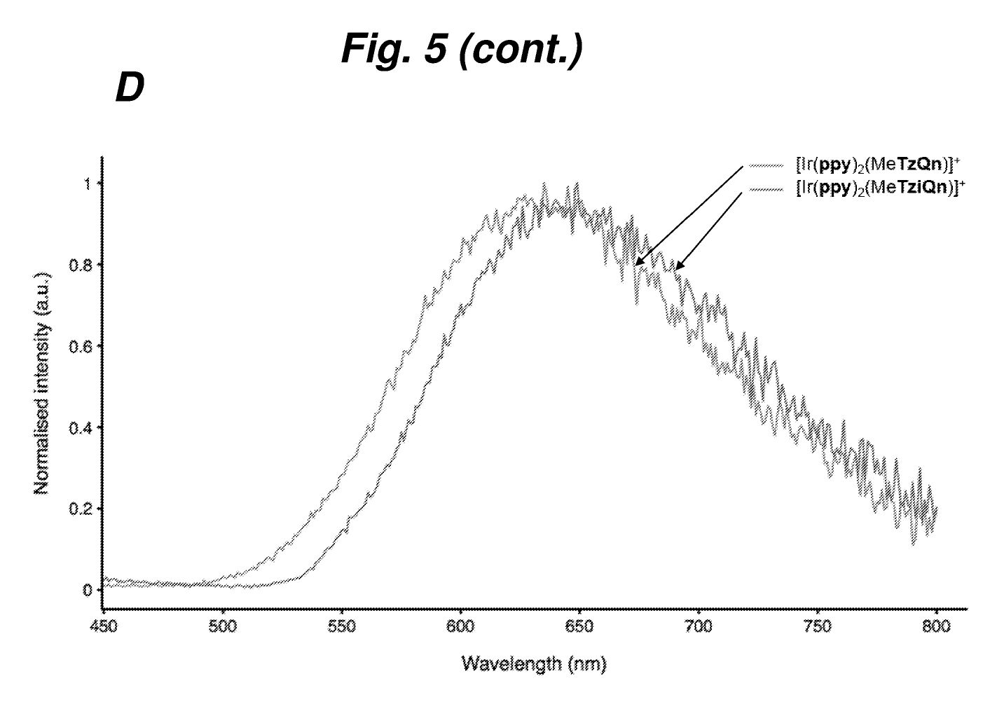

The emission spectra of the complexes from diluted dichloromethane solutions are reported in FIG. 5. The neutral complexes [Ir(ppy)₂(TzPyCN)], [Ir(F₂ppy)₂(TzPyCN)], and [Ir(ppy)₂(TzPyPhCN)] are characterised by broad and featureless emission bands, typical of radiative decay from excited states of charge transfer character. [Ir(ppy)₂(TzPyCN)] presents the most red-shifted maximum at 618 nm, which is ascribed to a stabilisation of the LUMO orbitals localised on the TzPyCN-ligand and associated to the electron-withdrawing nature of the nitrile functional group. As expected, the addition of electron-withdrawing fluoride substituents on the ppy ligands caused a blue-shift of the emission maximum for [Ir(F₂ppy)₂(TzPyCN)] to 542 nm, as a consequence of the stabilisation on the HOMO orbitals composing the LLCT(F₂ppy→*TzPyCN)/MLCT(Ir→*TzPyCN) transition. The emission maximum of [Ir(ppy)₂(TzPyPhCN)] appears blue-shifted to a lesser extent to 560 nm, which is ascribed to the fact that the electron-withdrawing effect of the nitrile group on the pyridyltetrazole π* system is decreased by the addition of a phenyl ring spacer twisted from co-planarity with respect to the pyridine ring.

The emission profiles of the methylated complexes [Ir(ppy)₂(MeTzPyCN)]⁺, [Ir(F₂ppy)₂(MeTzPyCN)]⁺, and [Ir(ppy)₂(MeTzPyPhCN)]⁺ is in all cases red-shifted compared to their neutral starting complexes. This red-shift is again evidence that the π* system of the pyridyl tetrazole ligand is involved in the charge transfer transitions. On comparing the emission maxima of the three methylated complexes, the observed trend is analogous to their neutral counterparts, with the most red-shifted emission originating from [Ir(ppy)₂(MeTzPyCN)]⁺.

The complexes containing quinoline and isoquinoline-functionalised tetrazolate ligands, [Ir(ppy)₂(TzQn)] and [Ir(ppy)₂(TziQn)], presents emission bands that cover a similar spectral range. Compared to the previously published complex [Ir(ppy)₂(TzPy)], where TzPy⁻ is the 5-(2′-pyridyl)tetrazolate ligand, showing a structured emission with λₑₘ=481 and 510 nm, [Ir(ppy)₂(TzQn)] and [Ir(ppy)₂(TziQn)] present a red-shifted emission that is ascribed to the increased it conjugation on passing from a phenyl to a quinolyl or isoquinolyl substituent. The increase in conjugation stabilises the π* system of the ligand and hence lowers the energy of the corresponding charge transfer transition. The structures of these two bands appear quite different. The emission profile of [Ir(ppy)₂(TzQn)] is broad and structureless, whereas that of [Ir(ppy)₂(TziQn)] appears structured. These results would suggest that there is a dominating charge transfer character for the emission of [Ir(ppy)₂(TzQn)]. On the other hand, the excited state of [Ir(ppy)₂(TziQn)] seems to be strongly influenced by LC character. TD-DFT calculations on these complexes (see SI) show that the lower energy bands are mainly composed of MLCT and LLCT transitions, with the difference from a contribution from the HOMO-4→LUMO transition of higher oscillator strength for [Ir(ppy)₂(TziQn)], which corresponds to a LC transition localised on the TziQn⁻ ligand. As expected and in line with the previously described complexes, the emission profiles of [Ir(ppy)₂(MeTzQn)]⁺ and [Ir(ppy)₂(MeTziQn)]⁺ appear red-shifted, broad, and structureless.

In all cases, upon degassing of the solution an elongation of the excited state lifetime and increase of quantum yield values are observed. This behaviour is indicative of the triplet spin multiplicity of the excited state.

An analysis of the variation of the emission bands in various solvents reveals solvatochromic behaviour, with a trend showing a red-shift of the emission maxima upon increasing the polarity of the solvent. In general, the most blue-shifted emission is recorded in ethyl laurate, and emission from dichloromethane are red-shifted by about 20-30 nm (with the exception of [Ir(ppy)₂(MeTzPyPhCN)]⁺ and [Ir(ppy)₂(MeTziQn)]⁺, for which almost no shift is observed). The bands further shift towards longer wavelength in aqueous media of about 10-30 nm. There are no significant variations without the various aqueous solvents, aside from [Ir(ppy)₂(MeTzPyPhCN)]⁺ that shows a 22 nm red-shift on passing from a PBS to a lysosomal fluid solution. The lack of significant variation suggests that it might be difficult to use this family of iridium tetrazolato probes for pH sensing in cells.

The methylated complex [Ir(ppy)₂(MeTzPyCN)]⁺ was found to have appreciable emission only in dichloromethane solution. Notably, the emission profile of [Ir(ppy)₂(TziQn)] progressively loses its vibrational structure on passing from organic to aqueous media. This effect is ascribed to the higher degree of solvatochromic nature for charged transfer transitions with respect to LC transitions, which causes the red-shifted emission in aqueous media to have a more dominant MLCT/LLCT component.

Lipophilicity

The lipophilicity of the iridium complexes was measured by the shake-flask method using 7.4-buffered PBS and n-octanol as solvents (Table 2). The values range between 1.49 and 2.68 are typical for cyclometalated iridium complexes that have not been bioconjugated. The only exception is represented by the methylated complex [Ir(ppy)₂(MeTzPyCN)]⁺, which shows a notably lower lipophilicity respect to the rest of the series of complexes. In general, neutral complexes have log D_{7.4} values above 2.00 and there is an increased in lipophilicity of extending the tetrazolato ligand with the addition of a phenyl spacer in [Ir(ppy)₂(TzPyPhCN)], or by extending the conjugation on passing from pyridine to quinoline or isoquinoline for [Ir(ppy)₂(TzQn)] and [Ir(ppy)₂(TziQn)]. Methylation in all cases reduces lipophilicity to log D_{7.4} values below 1.00, with the same trend within the family of methylated complexes as previously observed for the neutral ones.

TABLE 2
Distribution coefficient values (logD_{7.4}) for the complexes
obtain from 7.4-buffered PBS solution and n-octanol.

| Complex | logD_{7.4} |
|---|---|
| [Ir(ppy)₂(TzPyCN)] | 2.09 ± 0.06 |
| [Ir(F₂ppy)₂(TzPyCN)] | 2.01 ± 0.05 |
| [Ir(ppy)₂(TzPyPhCN)] | 2.68 ± 0.08 |
| [Ir(ppy)₂(MeTzPyCN)]⁺ | 0.64 ± 0.03 |
| [Ir(F₂ppy)₂(MeTzPyCN)]⁺ | 1.86 ± 0.02 |
| [Ir(ppy)₂(MeTzPyPhCN)]⁺ | 1.87 ± 0.08 |
| [Ir(ppy)₂(TzQn)] | 2.23 ± 0.04 |
| [Ir(ppy)₂(TziQn)] | 2.57 ± 0.05 |
| [Ir(ppy)₂(MeTzQn)]⁺ | 1.68 ± 0.05 |
| [Ir(ppy)₂(MeTziQn)]⁺ | 1.49 ± 0.06 |

Live H9c2 rat cardiomyoblasts were incubated with the iridium complexes for 30 minutes. Complexes were made to a final concentration of 20 μM in the cell culture medium with DMSO concentration <0.2%. Under these conditions, the luminescence from the iridium complexes within the cells was detected via confocal microscopy under single-photon excitation (403 nm) or two-photon excitation (810-840 nm).

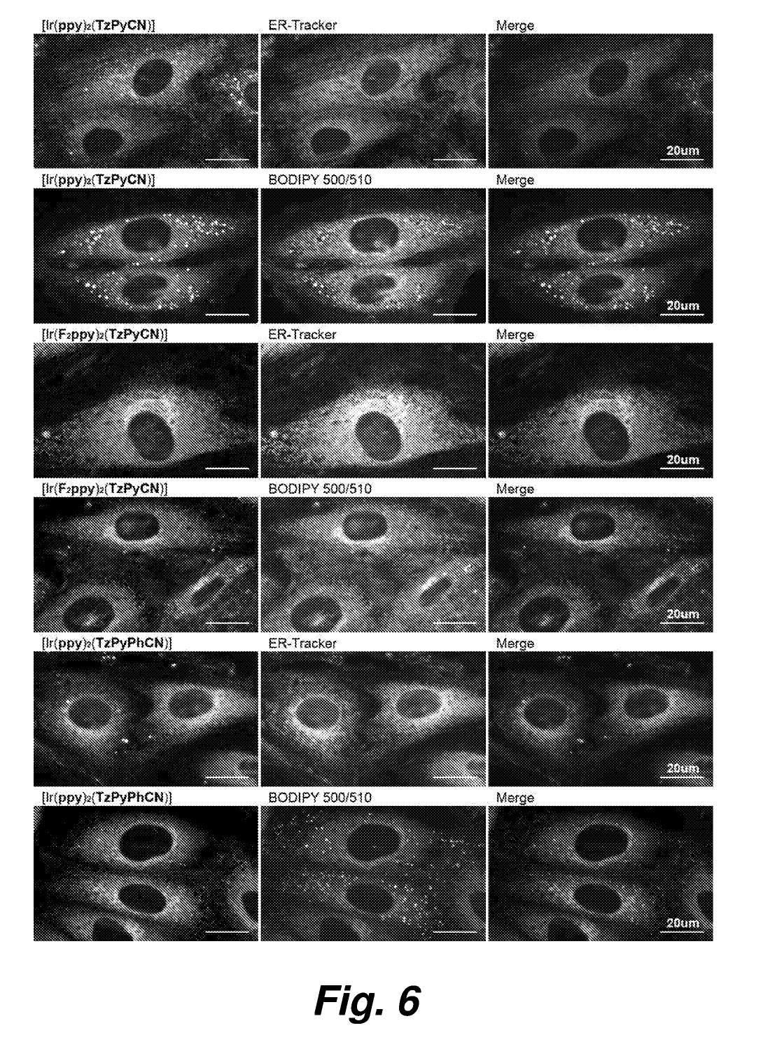

To determine the localisation of the iridium complexes, co-staining experiments were carried out with the use of ER-Tracker™ Red (endoplasmic reticulum), LysoTracker® Red DND-99 (acidic compartments), MitoTracker® Red (mitochondria), BODIPY® 500/510 C₁, C₁₂(fatty acid) or β-BODIPY® FL HPC (phospholipids). FIGS. 6-8 report the staining and co-staining experiments for all the complexes with the exception of [Ir(ppy)₂(MeTzPyCN)]⁺, for which no emission was detected from stained cells and this was in agreement with the reported photophysical data for this complex in aqueous media. The neutral complexes displayed brighter emission compared to their corresponding cationic methylated complexes under the same imaging conditions. This difference was also in agreement with the trend of the quantum yield data illustrated before and detection of internalised complex by ICP-MS. On the other hand, the solubility of the neutral complexes was found to be lower, as it can be seen in micrographs showing the occasional luminescence coming from precipitated complexes.

In general, all the neutral complexes displayed strong co-localisation with ER-Tracker™ and BODIPY® 500/510 C₁, C₁₂, suggesting co-localisation of these complexes to the endoplasmic reticulum and compartments which accumulate fatty acids such as lipid droplets. Therefore, changing the chemical nature of the ppy ligand by fluorination, adding a phenyl linker between the pyridine ring and CN moiety, and changing the pyridine ring for either a quinoline or isoquinoline substituent does not seem to influence significantly the cellular localisation of these neutral iridium complexes.

The methylated complexes [Ir(F₂ppy)₂(MeTzPyCN)]⁺ and [Ir(ppy)₂(MeTzPyPhCN)]⁺ evidence a staining pattern consistent with mitochondrial localisation, as highlighted by the co-staining experiments with MitoTracker® Red CMXRos. The lower affinity for the endoplasmic reticulum and lipid droplets could be ascribed to a reduced lipophilicity of the complexes. Moreover, the positive charge might be favouring crossing of the mitochondrial membrane as a consequence of the intermembrane electrical potential. This explanation would be consistent with the typical mitochondrial affinity of delocalised lipophilic cations, such as phosphonium salts, or previously reported cationic metal complexes. However, despite being less lipophilic than [Ir(F₂ppy)₂(MeTzPyCN)]⁺ and [Ir(ppy)₂(MeTzPyPhCN)]⁺, the (iso)quinoline complexes [Ir(ppy)₂(MeTzQn)]⁺ and [Ir(ppy)₂(MeTziQn)]⁺ showed a staining pattern more in line with their neutral analogues, with preferential localisation in the endoplasmic reticulum and fatty acids and phospholipids.

The emission profiles of all the neutral complexes from the cells were consistent with the emission profiles recorded in aqueous PBS solution (see SI). Interestingly, the analysis of the emission profiles from cells incubated with the cationic complexes Ir(F₂ppy)₂(MeTzPyCN)]⁺, [Ir(ppy)₂(MeTzPyPhCN)]⁺, and [Ir(ppy)₂(MeTzQn)]⁺ revealed blue-shifted and almost superimposable bands between these cationic complexes and their neutral analogues. This evidence was consistent analysing the emission profiles both under one-photon or two-photon excitation (see SI). The only complex that did not show this change was [Ir(ppy)₂(MeTziQn)]⁺, for which two distinct emission profiles were recorded on comparing cells stained with this complex and its neutral analogue, with emission from [Ir(ppy)₂(MeTziQn)]⁺ being red-shifted consistently with the photophysical data.

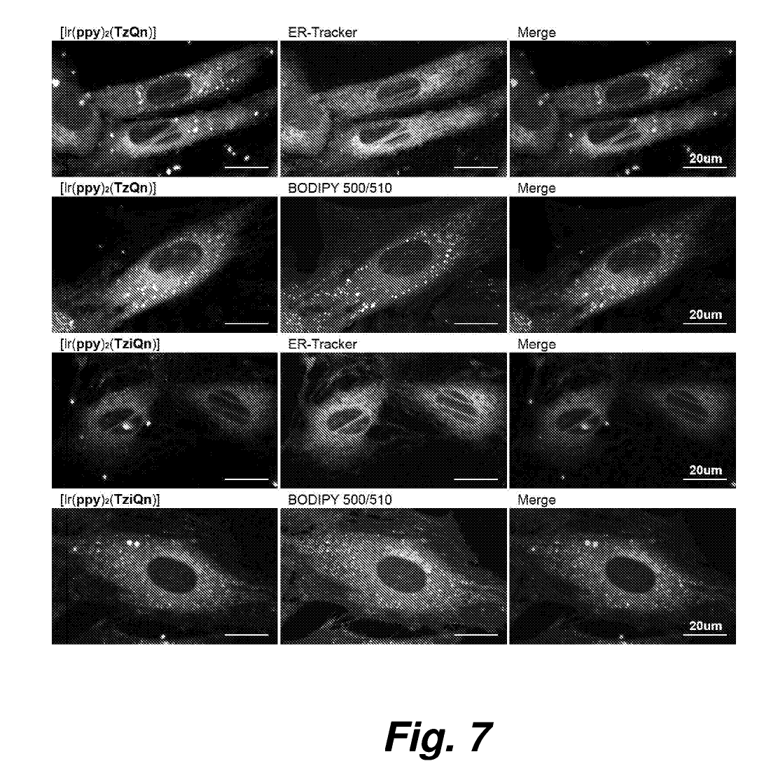

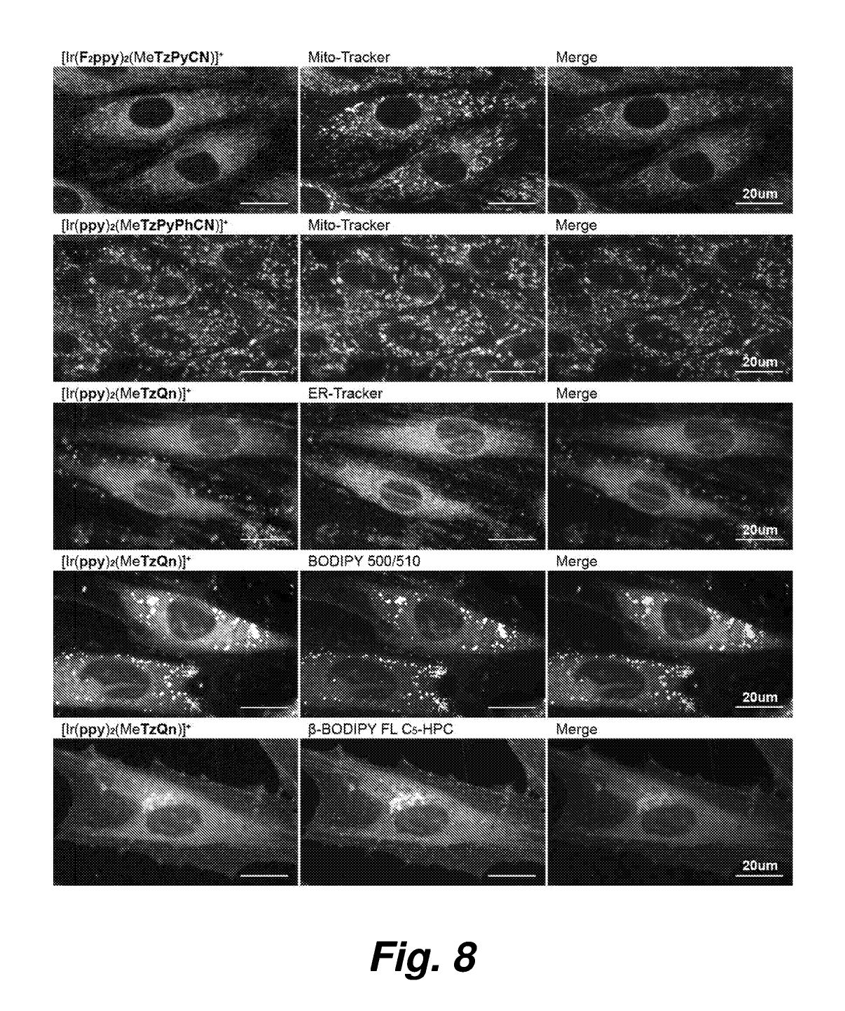

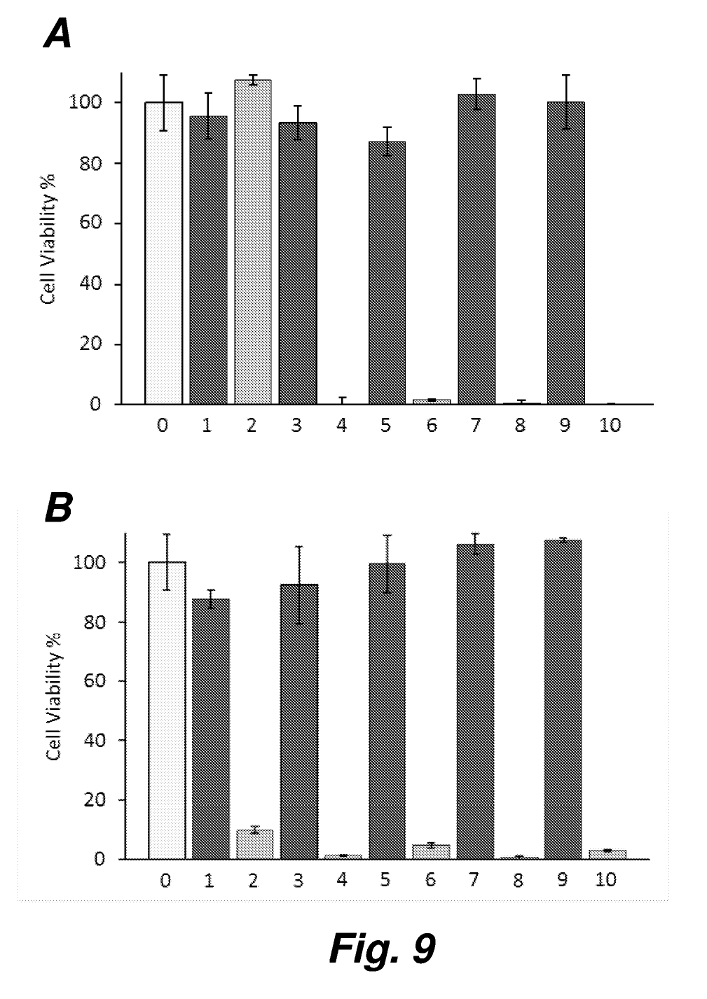

The cellular viability after the incubation with the iridium complexes was assessed via MTS assay. Cells were incubated with the complexes at a concentration of 20 and 40 μM for 24 hours to assess long term cytotoxic effect and for 4 hours to see the effect of a shorter time point. The cell viability plots are reported in FIG. 9. From the results, it is immediately obvious that, in general, neutral iridium complexes are less cytotoxic than cationic iridium complexes when cells were exposed for 24 hours. These findings are aligned with the majority of reported cationic iridium complexes, which were found to be quite cytotoxic. After 4 hours cells exposed to cationic iridium complexes showed some cytotoxic effect however these were not as pronounces as those observed at 24 hours. This indicates that short exposure time to these complexes has less cytotoxic effects than long. Remarkably, this series of iridium tetrazolato complexes has proven to be a promising candidate for the imaging of the endoplasmic reticulum without significant cytotoxic effect, contrary to other examples of iridium complexes that were shown to accumulate in the endoplasmic reticulum.

### EXPERIMENTAL SECTION

General Considerations

Unless otherwise stated, all reagents and solvents were purchased from Sigma Aldrich or Alfa Aesar and used as received without further purification. Precursors for HTzPyCN^{1,2}, HTzQn^{3,4}, HTziQn⁵, [Ir(ppy)₂(μ-Cl)]₂⁶, [Ir(F₂ppy)₂(μ-Cl)]₂^{6,7} and [Ir(ppy)₂(TzPyBr)]⁸ were prepared according to previously published procedures.

Nuclear magnetic resonance spectra were recorded using a Bruker Avance 400 spectrometer (400 MHz for ¹H-NMR; 100 MHz for 13C-NMR) at 300 K. All the NMR spectra were calibrated to residual solvent signals. Proton and carbon atoms were described as in FIG. 10 as pyridinic (A), phenylic (B) and tetrazolic (T) ones. Infrared spectra were recorded using an attenuated total reflectance Perkin Elmer Spectrum 100 FT-IR with a diamond stage. IR spectra were recorded from 4000-650 cm⁻¹. The intensity of the band are reported as strong (s), medium (m), or weak (w), with broad (br) bands also specified. Melting points were determined using a BI Barnsted Electrotermal 9100 apparatus. Elemental analysis were obtain using a Thermo Finning EA 1112 Series Flash.

Synthesis of the Ligands and Complexes

HTzPyCN. 2,5-dicyanopyridine (0.100 g, 0.775 mmol), sodium azide (0.050 g, 0.775 mmol) and zinc bromide (0.192 g, 0.853 mmol) were dissolved in 10 mL of water. The resulting suspension was vigorously stirred and heated at reflux overnight. The milky reaction mixture was then made basic by addition of a 0.25 M sodium hydroxide solution (7.76 mL, 1.938 mmol) and the formed yellow precipitate was filtered. The filtrate was acidified to pH≈1 with 3 M hydrochloric acid. The formed white precipitate was then filtered and dried in air. Yield: 0.085 g (64%), M.P. 234° C. (dec). IR (ν/cm⁻¹): 2235 w (C≡N), 1604 w (tetrazole C═N). ¹H NMR (δ/ppm, DMSO-d⁶): 9.27 (s, 1H, H₆), 8.59 (dd, 1H, H₄, J=8.2 Hz, J′=2.1 Hz), 8.38 (dd, 1H, H₃, J=8.2 Hz, J′=0.8 Hz). ¹³C NMR (δ/ppm, DMSO-d⁶): 154.4 (Cₜ), 153.0 (CH₆), 146.6 (CN), 142.1 (CH₄), 122.4 (CH₃), 116.5 (C₅), 110.6 (C₂). Anal. Calcd for HTzPyCN⋅0.45(H₂O)⋅0.09(bis tetrazole): C, 45.54; H, 2.32; N, 47.40. Found: C, 45.88; H, 2.70; N, 47.77.

[Ir(ppy)₂(TzPyCN)]. [Ir(ppy)₂(μ-Cl)]₂ (0.226 g, 0.211 mmol) was combined with HTzPyCN (0.080 g, 0.465 mmol) and dissolved in 13 mL of a dichloromethane/ethanol mixture (10:3 v/v). The resulting suspension was stirred at room temperature overnight. The solvents were concentrated and the product was purified via column chromatography using Brockmann I grade neutral alumina-filled as stationary phase and a dichloromethane/acetone (9:1 v/v) solvent system as eluent. The targeted complex eluted as the second fraction (yellow). Yield: 0.215 g (74%). M.P. 210° C. (dec.). IR (ν/cm⁻¹): 2235 w (C≡N), 1606 w (tetrazole C═N). ¹H NMR (δ/ppm, acetone-d⁶): 8.51 (ddd, 2H, H_{T4}, H_{T3}, J=13.4 Hz, J′=8.4 Hz, J″=1.2 Hz), 8.18-8.12 (m, 2H, 2H_{A}), 8.12 (s, 1H, H_{T6}), 7.93-7.88 (m, 2H, 2H_{A}), 7.86 (dd, 2H, 2H_{A}, J=8.0 Hz, J′=1.6 Hz), 7.77 (dd, 1H, H_{B}, J=7.6 Hz, J′=1.6 Hz), 7.58 (ddd, 1H, H_{B}, J=5.8 Hz, J′=1.6 Hz, J″=0.8 Hz), 7.17 (ddd, 1H, H_{B}, J=7.3 Hz, J′=5.8 Hz, J″=1.4 Hz), 7.06-6.99 (m, 2H, 2H_{A}), 6.93-6.88 (m, 2H, 2H_{B}), 6.78 (td, 1H, H_{B}, J=7.4 Hz, J′=1.4 Hz), 6.43 (dd, 1H, H_{B}, J=7.5 Hz, J′=0.8 Hz), 6.29 (dd, 1H, H_{B}, J=7.7 Hz, J′=0.7 Hz). ¹³C NMR (6/ppm, acetone-d⁶): 168.9 (C_{A}), 168.8 (C_{A}), 163.7 (C_{T}), 153.9 (CH_{T}), 153.5 (C_{T}), 152.0 (C_{B}), 150.4 (CH_{A}), 147.4 (C_{B}), 145.7 (C_{B}), 145.0 (C_{B}), 143.5 (CH_{T}), 139.2 (CH_{A}), 138.8 (CH_{A}), 132.9 (CH_{B}), 132.5 (CH_{B}), 131.0 (CH_{B}), 130.1 (CH_{B}), 125.7 (CH_{A}), 125.1 (CH_{B}), 124.3 (CH_{B}), 123.8 (CH_{B}), 123.2 (2CH_{A}), 123.1 (CH_{T}), 122.4 (CH_{B}), 120.5 (CH_{A}), 120.1 (CH_{A}), 116.1 (C_{T}), 112.3 (C_{T}). Anal. Calcd for [Ir(ppy)₂(TzPyCN)]⋅0.75(acetone): C, 52.47; H, 3.31; N, 15.67. Found: C, 52.79; H, 2.95; N, 15.71.

[Ir(F₂ppy)₂(TzPyCN)]. [Ir(F₂ppy)₂(μ-Cl)]₂ (0.129 g, 0.106 mmol) was combined with HTzPyCN (0.040 g, 0.233 mmol) and dissolved in 13 mL of a dichloromethane/ethanol mixture (10:3 v/v). The resulting suspension was stirred at room temperature overnight. The solvents were concentrated and the product was purified via column chromatography using Brockmann I grade neutral alumina-filled as stationary phase and a dichloromethane/acetone (9:1 v/v) solvent system as eluent. The targeted complex eluted as the second fraction (yellow). Yield: 0.055 g (70%). M.P. 252° C. (dec). IR (ν/cm⁻¹): 2232 w (C≡N), 1601 w (tetrazole C═N). ¹H NMR (δ/ppm, acetone-d⁶): 8.51 (dd, 2H, H_{T4}, H_{T3}, J=8.4 Hz, J′=1.6 Hz), 8.33 (s, 1H, H_{T6}), 8.28 (t, 2H, H_{A}, J=8.5 Hz), 8.00-7.91 (m, 3H, 3H_{A}), 7.51 (d, 1H, H_{A}, J=5.8 Hz), 7.23 (ddd, 1H, H_{A}, J=7.4 Hz, J′=5.8 Hz, J″=1.4 Hz), 7.10 (ddd, 1H, H_{A}, J=7.4 Hz, J′=5.8 Hz, J″=1.4 Hz), 6.66 (ddd, 1H, H_{B}, J=12.4 Hz, J′=2.4 Hz, J″=0.8 Hz), 6.59 (ddd, 1H, H_{B}, J=12.4 Hz, J′=2.4 Hz, J″=0.8 Hz), 5.84 (dd, 1H, H_{B}, J=8.6 Hz, J′=2.4 Hz), 5.66 (dd, 1H, H_{B}, J=8.9 HZ, J′=2.4 Hz). ¹³C NMR (δ, ppm, acetone-d⁶): 165.7 (d, 2C_{B}, J_{CF}=50.4 Hz), 165.2 (d, C_{B}, J_{CF}=28.8 Hz), 165.0 (d, C_{B}, J_{CF}=28.8 Hz), 163.6 (2C_{A}), 163.2 (2C_{B}), 163.1 (d, 2C_{B}, J_{CF}=20.0 Hz), 154.7 (CH_{T}), 152.8 (C_{T}), 151.6 (d, 2CH_{A}, J_{CF}=28.0 Hz), 150.8 (d, CH_{A}, J_{CF}=107.2 Hz), 144.3 (CH_{T}), 140.2 (d, CH_{A}, J_{CF}=111.6 Hz), 129.5 (d, CH_{B}, J_{CF}=15.2 Hz), 128.9 (d, CH_{B}, J_{CF}=17.6 Hz), 124.8 (CH_{A}), 124.4 (CH_{A}), 124.0 (t, CH_{A}, J_{CF}=97.2 Hz), 123.4 (CH_{T}), 115.9 (C_{T}), 114.9 (dd, CH_{B}, J_{CF}=72.0 Hz, J_{CCF}=11.6 Hz), 112.8 (C_{T}), 99.5 (t, CH_{B}, J_{CF}=108 Hz), 98.7 (t, CH_{B}, J_{CF}=108 Hz); quaternary tetrazolic C peak was not visible in the spectrum. Crystals suitable for X-ray analysis were obtained by slow diffusion of hexane into a solution of the complex in dichloromethane. Anal. Calcd for [Ir(F₂ppy)₂(TzPyCN)]⋅0.2(CH₂Cl₂): C, 46.39; H, 2.13; N, 14.62. Found: C, 46.36; H, 1.75; N, 14.68.

[Ir(ppy)₂(TzPyPhCN)]. [Ir(ppy)₂(TzPyBr)] (0.150 g, 0.204 mmol), 4-cyanophenylboronic acid (0.036 g, 0.248 mmol), and bis(triphenylphosphine)palladium(II) dichloride (0.005 g, 0.006 mmol) were combined and dissolved in 10 mL of dry THF. The solution was stirred under nitrogen for 15 minutes and 1 M aqueous Na₂CO₃ (15 mL, 0.290 mmol) was added and refluxed overnight. The cooled crude mixture was washed with water and extracted with dichloromethane (3×15 mL). The combined organic phase was dried over MgSO₄ and filtered. The product was purified via column chromatography using Brockmann I grade neutral alumina-filled as stationary phase and a dichloromethane/acetone (9:1 v/v) solvent system as eluent. The targeted complex eluted as the second fraction (yellow). Yield: 0.134 g (87%). M.P. 234-236° C. IR (ν/cm⁻¹): 2232 w (C≡N), 1606 w (tetrazole C═N). 1H NMR (δ/ppm, DMSO-d⁶): 8.51 (dd, 1H, H_{T4}, J=8.2 Hz, J′=2.0 Hz), 8.44 (d, 1H, H_{T3}, J=8.2 Hz), 8.20 (t, 2H, 2H_{A}, J=8.4 Hz), 7.94-7.83 (m, 6H, 2H_{Tph}, 3H_{B}, H_{A}), 7.77 (d, 1H, H_{A}, J=7.6 Hz), 7.64-7.60 (m, 1H, H_{T6}), 7.52 (d, 2H, 2H_{Tph}, J=8.4 Hz), 7.46 (d, 1H, H_{A}, J=7.6 Hz), 7.19 (t, 1H, H_{A}, J=7.2 Hz), 7.10 (t, 1H, H_{A}, J=7.2 Hz), 7.02 (t, 1H, H_{A}, J=8.2 Hz), 6.93 (t, 2H, 2H_{B}, J=7.4 Hz), 6.79 (t, 1H, H_{B}, J=7.6 Hz), 6.32 (d, 1H, H_{B}, J=6.4 Hz), 6.20 (d, 1H, H_{B}, J=6.4 Hz). ¹³C NMR (δ/ppm, acetone-d⁶): 167.2 (C_{A}), 167.0 (C_{A}), 163.1 (C_{T}), 151.5 (C_{B}), 149.2 (CH_{A}), 148.9 (CH_{A}), 147.9 (C_{T}), 147.4 (CH_{B}), 147.2 (CH_{B}), 144.5 (C_{B}), 144.1 (C_{B}), 139.4 (C_{T}), 138.5 (CH_{A}), 138.2 (CH_{A}), 138.0 (CH_{T}), 136.2 (C_{T}), 133.2 (CH_{T}), 131.4 (CH_{T}), 131.5 (CH_{B}), 130.0 (CH_{B}), 129.1 (CH_{B}), 128.8 (CH_{B}), 128.7 (CH_{B}), 127.4 (CH_{T}), 124.9 (C_{B}), 124.4 (CH_{T}), 123.7 (CH_{A}), 123.4 (CH_{A}), 122.4 (CH_{T}), 122.0 (CH_{T}), 121.2 (CH_{B}), 119.8 (CH_{A}), 119.3 (CH_{A}), 118.3 (C_{T}), 111.7 (C_{T}). Anal. Calcd for [Ir(ppy)₂(TzPyPhCN)]⋅1(CH₂Cl₂)⋅0.33(acetone): C, 52.15; H, 3.19; N, 13.15. Found: C, 51.99; H, 2.82; N, 13.37. [Ir(ppy)₂(MeTzPyCN)][PF₆]. [Ir(ppy)₂(TzPyCN)] (0.060 g, 0.089 mmol) was dissolved in (10 mL) and cooled down to −50° C. using an ethyl acetate/liquid nitrogen cool bath. Thereafter, a 0.1 M methyl trifluoromethanesulfonate solution in DCM (0.022 g, 0.133 mmol) was added dropwise to the vigorously stirred solution. After being maintained at −50° C. for 30 minutes, the solution was warmed up at room temperature and left to stirred overnight. An excess of ammonium hexafluorophosphate (0.029 g, 0.178 mmol) was added and stirred for 45 minutes. Then the product was extracted with dichloromethane and water (3×15 mL) and the combined organic phase was dried on MgSO₄. The targeted complex was then collected after filtration and removal of the solvent as a red solid. Yield: 0.063 g (85%). M.P. 242-243° C. IR (ν/cm⁻¹): 2240 w (C≡N), 1609 w (tetrazole C═N). ¹H NMR (δ/ppm, aceton^{e}-d⁶): 8.79 (dd, 1H, H_{T4}, J=8.2 Hz, J′=0.9 Hz), 8.73 (dd, 1H, H_{T3}, J=8.2 Hz, J′=1.9 Hz), 8.32 (s, 1H, H_{T6}), 8.22 (dd, 2H, 2H_{A}, J=8.0 Hz, J′=3.5 Hz), 8.03-7.96 (m, 4H, 4H_{A}), 7.89 (dd, 1H, H_{B}, J=7.7 Hz, J′=1.4 Hz), 7.83 (dd, 1H, H_{B}, J=7.7 Hz, J′=1.4 Hz), 7.14 (tdd, 2H, 2H_{A}, J=7.5 Hz, J′=5.8 Hz, J″=1.4 Hz), 7.06 (td, 1H, H_{B}, J=7.8 Hz, J′=1.2 Hz), 9.69 (dtd, 2H, 2H_{B}, J=15.5 Hz, J′=7.4 Hz, J″=1.3 Hz), 6.85 (td, 1H, H_{B}, J=7.5 Hz, J′=1.4 Hz), 6.34 (dd, 1H, H_{B}, J=7.6 Hz, J′=0.8 Hz), 6.28 (dd, 1H, H_{B}, J=7.7, J′=0.8 Hz), 4.57 (s, 3H, CH_{TMe}). ¹³C NMR (δ/ppm, acetone-d⁶): 1680.5 (C_{A}), 1680.1 (C_{A}), 1660.8 (C_{T}), 1540.9 (CH_{T}), 151.2 (CH_{A}), 1510.1 (CH_{A}), 1480.6 (C_{T}), 1470.5 (C_{B}), 145.3 (C_{B}), 145.2 (C_{B}), 144.9 (CH_{T}), 144.0 (C_{B}), 139.9 (CH_{A}), 139.8 (CH_{A}), 132.7 (CH_{B}), 132.4 (CH_{B}), 131.3 (CH_{B}), 130.6 (CH_{B}), 126.0 (CH_{B}), 125.6 (CH_{B}), 125.4 (CH_{T}), 124.6 (CH_{A}), 124.4 (CH_{A}), 124.1 (CH_{B}), 123.5 (CH_{B}), 121.0 (CH_{A}), 120.6 (CH_{A}), 115.9 (C_{T}), 115.5 (C_{T}), 42.7 (CH_{TMe}). Crystals were formed in the NMR tube and have been used for the elemental analysis. Anal. Calcd for [Ir(ppy)₂(MeTzPyCN)][PF₆]⋅0.3(CH₂Cl₂)⋅0.3(acetone): C, 43.26; H, 2.88; N, 12.81. Found: C, 43.21; H, 2.57; N, 12.77.

[Ir(F₂ppy)₂(MeTzPyCN)][PF₆]. [Ir(F₂ppy)₂(TzPyCN)] (0.060 g, 0.081 mmol) was dissolved in DCM (10 mL) and cooled down to −50° C. using an ethyl acetate/liquid nitrogen cool bath. A 0.1 M methyl trifluoromethanesulfonate solution in DCM (0.020 g, 0.121 mmol) was added dropwise to the vigorously stirred solution. After being maintained at −50° C. for 30 minutes, the solution was warmed up at room temperature and left to stir overnight. An excess of ammonium hexafluorophosphate (0.009 g, 0.162 mmol) was added and stirred for 45 minutes. Then the product was extracted with dichloromethane and water (3×15 mL) and the combined organic phase was dried on MgSO₄. The targeted complex was then collected after filtration and removal of the solvent as a red solid. Yield: 64.4 g (88%). M.P. 293° C. (dec.). IR (ν/cm⁻¹): 2243 w (C≡N), 1599 w (tetrazole C═N). ¹H NMR (δ/ppm, acetone-d⁶): 8.84 (ddd, 2H, H_{T4}, H_{T3}, J=8.8 Hz, J′=8.0 Hz, J″=0.8 Hz), 8.63 (s, 1H, H_{T6}), 8.37 (dd, 2H, 2H_{A}, J=8.8 Hz, J′=1.6 Hz), 8.11-8.06 (m, 4H, 4H_{A}), 7.26-7.21 (m, 4H, 2H_{A}), 6.76 (dddd, 2H, 2H_{B}, J=15.3 Hz, J′=12.7 Hz, J″=9.3 Hz, J′″=2.4 Hz), 5.79 (dd, 1H, H_{B}, J=8.6 Hz, J′=2.3 Hz), 5.71 (dd, 1H, H_{B}, J=8.7 Hz, J′=2.3 Hz), 4.61 (s, 3H, CH_{TME}). ¹³C NMR (δ/ppm, acetone-d⁶): 166.7 (C_{T}), 165.5 (d, C_{B}, J_{CF}=12.8 Hz), 165.0 (d, C_{B}, J_{CF}=12.6 Hz), 163.4 (d, C_{A}, J_{CF}=13.1 Hz), 163.0 (d, C_{B}, J_{CF}=20.8 Hz), 162.9 (d, C_{B}, J_{CF}=21.2 Hz), 162.4 (d, C_{B}, J_{CF}=12.5 Hz), 160.9 (d, C_{B}, J_{CF}=12.9 Hz), 160.3 (d, C_{B}, J_{CF}=52.0 Hz), 155.8 (CH_{T}), 151.7 (CH_{A}), 151.5 (CH_{A}), 151.2 (d, C_{A}, J_{CF}=27.6 Hz), 148.0 (C_{T}), 145.7 (CH_{T}), 141.0 (2CH_{A}), 129.2 (d, C_{B}, J_{CF}=74.0 Hz), 125.8 (CH_{T}), 125.1 (d, 2CH_{A}, J_{CF}=30.8 Hz), 124.7 (d, CH_{A}, J_{CF}=78.8 Hz), 124.3 (d, CH_{A}, J_{CF}=80.4 Hz), 116.3 (C_{T}), 115.3 (C_{T}), 114.9 (t, CH_{B}, J_{CF}=14.8 Hz), 114.8 (t, CH_{B}, J_{CF}=12.8 Hz), 100.4 (t, CH_{B}, J_{CF}=107.6 Hz), 99.9 (t, CH_{B}, J_{CF}=92.0 Hz), 42.9 (CH_{TMe}). Crystals not suitable for X-ray analysis were obtained by slow diffusion of diethyl ether into a solution of the complex in dichloromethane. They have been used for elemental analysis. Anal. Calcd for [Ir(F₂ppy)₂(MeTzPyCN)][PF₆]⋅0.0.25(diethyl ether): C, 40.37; H, 2.24; N, 12.15. Found: C, 40.20; H, 1.95; N, 11.91.

[Ir(ppy)₂(MeTzPyMeCN)][PF₆]. [Ir(ppy)₂(TzPyPhCN)] (0.050 g, 0.067 mmol) was dissolved in DCM (10 mL) and cooled down to −50° C. using ethyl acetate/liquid nitrogen cool bath. A 0.1M methyl trifluoromethanesulfonate solution in DCM (0.016 g, 0.100 mmol) was added dropwise to the vigorously stirred solution. After being maintained at −50° C. for 30 minutes, the solution was warmed up at room temperature and left to stir overnight. An excess of ammonium hexafluorophosphate (0.022 g, 0.134 mmol) was added and stirred for 45 minutes. Then the product was extracted with dichloromethane and water (3×15 mL) and the combined organic phase was dried on MgSO₄. The targeted complex was then collected after filtration and removal of the solvent as a yellow solid. Yield: 0.044 g (73%). M.P. 222-224° C. IR (ν/cm⁻¹): 2229 w (C≡N), 1608 w (tetrazole C═N). ¹H NMR (δ/ppm, DMSO-d⁶): 8.68 (dd, 2H, H_{T4}, H_{T3}, J=8.4 Hz, J′=2.0 Hz), 8.24 (d, 2H, 2H_{A}, J=9.2 Hz), 8.00-7.85 (m, 8H, H_{T6}, 4H_{Tph}, 3H_{A}), 7.58 (d, 3H, 3H_{B}, J=8.8 Hz), 7.21-7.14 (m, 2H, 2H_{A}), 7.06 (td, 1H, H_{A}, J=7.6 Hz, J′=1.6 Hz), 7.00-6.93 (m, 2H, 2H_{B}), 6.83 (td, 1H, H_{B}, J=7.6 Hz, J′=1.6 Hz), 6.23 (dd, 1H, H_{B}, J=4.2 Hz, J′=1.2 Hz), 6.16 (dd, 1H, H_{B}, J=4.2 Hz, J′=1.2 Hz), 4.54 (s, 3H, CH_{TMe}). ¹³C NMR (δ/ppm, DMSO-d⁶): 166.8 (C_{T}), 166.2 (C_{T}), 165.6 (C_{T}), 150.3 (CH_{A}), 150.1 (CH_{A}), 148.3 (CH_{T}), 147.6 (C_{B}), 144.2 (C_{A}), 144.1 (C_{A}), 144.0 (C_{T}), 143.5 (C_{T}), 139.1 (CH_{T}), 139.0 (CH_{T}), 138.9 (CH_{T}), 138.8 (CH_{T}), 133.3 (CH_{A}), 132.0 (CH_{B}), 131.5 (CH_{B}), 131.4 (CH_{B}), 130.9 (CH_{B}), 130.3 (CH_{B}), 129.5 (CH_{B}), 128.8 (C_{B}), 128.7 (C_{B}), 127.8 (CH_{B}), 125.1 (CH_{T}), 124.6 (CH_{T}), 124.0 (CH_{A}), 123.9 (CH_{A}), 122.8 (CH_{A}), 122.2 (CH_{B}), 120.1 (CH_{A}), 119.8 (CH_{A}), 118.2 (C_{T}), 112.3 (C_{B}), 42.1 (CH_{TMe}). Crystals suitable for X-ray analysis were obtained by slow diffusion of hexane into a solution of the complex in dichloromethane. Even after crystallisation, the separation of the complex from triphenylphosphine derived from the Suzuki reaction was impossible to achieve. Anal. Calcd for [Ir(ppy)₂(MeTzPyMeCN)][PF₆]⋅1(CH₂Cl₂)⋅0.33(PPh₃): C, 47.81; H, 3.08; N, 10.37. Found: C, 47.79; H, 3.03; N, 10.35.

[Ir(ppy)₂(TzQn)]. [Ir(ppy)₂(μ-Cl)]₂ (0.250 g, 0.233 mmol) was combined with HTzQn (0.115 g, 0.583 mmol) and dissolved in 13 mL of a dichloromethane/ethanol mixture (10:3 v/v). The resulting suspension was stirred at room temperature overnight. The solvents were concentrated and the product was purified via column chromatography using Brockmann I grade neutral alumina-filled as stationary phase and a dichloromethane/acetone (8:2 v/v) solvent system as eluent. The targeted complex eluted as the second fraction (yellow). Yield: 0.172 g (53%). M.P. 314-317° C. IR (ν/cm⁻¹): 1602 w (tetrazole C═N). ¹H NMR (δ/ppm, acetone-d⁶): 8.69 (d, 1H, H_{T4}, J=8.2 Hz), 8.54 (d, 1H, H_{T3}), 8.14 (d, 1H, H_{T9}), 8.10-7.98 (m, 4H, 4H_{A}), 7.86-7.73 (m, 4H, 4H_{B}), 7.54 (td, 1H, H_{A}, J=7.4 Hz, J′=1.2 Hz), 7.33 (dd, 1H, H_{A}, J=6.0 Hz, J′=0.8 Hz), 7.21 (td, 1H, H_{A}, J=8.0 Hz, J′=1.6 Hz), 7.06 (td, 1H, H_{A}, J=6.8 Hz, J′=1.6), 7.02-6.97 (m, 4H, H_{T6}, H_{T7}, H_{T8}, H_{B}), 6.73 (td, 1H, H_{B}, J=7.5 Hz, J′=1.4 Hz), 6.54 (dd, 1H, H_{B}, J=7.6 Hz, J′=1.1 Hz), 6.16 (dd, 1H, H_{B}, J=7.6 Hz, J′=0.8 Hz). ¹³C NMR (δ/ppm, acetone-d⁶): 169.5 (C_{A}), 168.7 (C_{A}), 166.0 (C_{T}), 155.5 (C_{B}), 152.5 (C_{T}), 151.3 (CH_{A}), 149.7 (CH_{A}), 148.7 (C_{T}), 145.6 (C_{B}), 144.7 (C_{B}), 144.6 (C_{B}), 141.6 (CH_{T}), 138.8 (CH_{B}), 138.5 (CH_{B}), 133.2 (CH_{B}), 131.9 (CH_{B}), 130.8 (CH_{A}), 130.2 (CH_{T}), 130.1 (CH_{T}), 129.8 (CH_{B}), 128.9 (CH_{A}), 128.5 (CH_{A}), 125.7 (CH_{B}), 124.9 (CH_{B}), 123.9 (CH_{A}), 123.6 (CH_{A}), 122.5 (CH_{T}), 122.4 (CH_{B}), 120.6 (CH_{T}), 120.2 (CH_{T}), 120.1 (CH_{A}); quaternary tetrazolic C peak was not visible in the spectrum. Anal. Calcd for [Ir(ppy)₂(TzQn)]⋅0.08(CH₂Cl₂)⋅0.75(ethanol)⋅0.83(acetone): C, 55.08; H, 4.06; N, 12.46. Found: C, 55.09; H, 3.94; N, 12.10.

[Ir(ppy)₂(TziQn)]. [Ir(ppy)₂(μ-Cl)]₂ (0.250 g, 0.233 mmol) was combined with HTzQn (0.115 g, 0.583 mmol) and dissolved in 13 mL of a dichloromethane/ethanol mixture (10:3 v/v). The resulting suspension was stirred at room temperature overnight. The solvents were concentrated and the product was purified via column chromatography using Brockmann I grade neutral alumina-filled as stationary phase and a dichloromethane/acetone (8:2 v/v) solvent system as eluent. The targeted complex eluted as the second fraction (yellow). Yield: 0.235 g (72%). M.P. 272-275° C. IR (ν/cm⁻¹): 1606 w (tetrazole C═N). ¹H NMR (δ/ppm, acetone-d⁶): 10.25-10.21 (m, 1H, H_{T9}), 8.16-8.12 (m, 2H, 2H_{A}), 8.10-8.04 (m, 1H, H_{T}10), 7.97-7.94 (m, 2H, H_{T6}, H_{T7}), 7.88-7.81 (m, 5H, 3H_{A}, H_{T4}, H_{T5}), 7.79-7.75 (m, 2H, H_{A}, H_{B}), 7.59 (dd, 1H, H_{B}, J=6.0 Hz, J′=0.8 Hz), 7.11 (td, 1H, H_{B}, J=6.8 Hz, J′=1.6 Hz), 7.04-6.98 (m, 2H, 2H_{A}), 6.94-6.89 (m, 2H, 2H_{B}), 6.78 (td, 1H, H_{B}, J=7.4 Hz, J′=1.2 Hz), 6.67 (dd, 1H, H_{B}, J=7.6 Hz, J′=0.8 Hz), 6.35 (dd, 1H, H_{B}, J=7.6 Hz, J′=0.8 Hz). ¹³C NMR (δ/ppm, acetone-d⁶): 169.3 (C_{A}), 169.1 (C_{A}), 166.0 (C_{T}), 154.7 (C_{T}), 150.4 (CH_{B}), 150.1 (CH_{B}), 149.6 (C_{B}), 145.6 (C_{B}), 145.1 (C_{B}), 142.5 (CH_{T}), 138.8 (CH_{A}), 138.5 (CH_{A}), 137.7 (C_{T}), 133.5 (C_{T}), 132.8 (CH_{B}), 132.7 (CH_{B}), 131.0 (CH_{B}), 130.4 (CH_{B}), 130.1 (CH_{T}), 128.9 (CH_{T}), 128.1 (CH_{T}), 127.3 (C_{B}), 125.6 (CH_{T}), 125.1 (CH_{A}), 125.0 (CH_{A}), 124.1 (CH_{B}), 123.7 (CH_{A}), 122.8 (CH_{A}), 122.1 (CH_{B}), 120.2 (CH_{A}), 120.1 (CH_{A}); quaternary tetrazolic C peak was not visible in the spectrum. Anal. Calcd for [Ir(ppy)₂(TziQn)]⋅0.67(CH₂Cl₂): C, 52.08; H, 3.12; N, 13.01. Found: C, 52.26; H, 2.94; N, 13.04.

[Ir(ppy)₂(MeTzQn)][PF₆]. [Ir(ppy)₂(TzQn)] (0.050 g, 0.072 mmol) was dissolved in (10 mL) and cooled down to −50° C. using an ethyl acetate/liquid nitrogen cool bath. Thereafter, a 0.1 M methyl trifluoromethanesulfonate solution in DCM (0.018 g, 0.108 mmol) was added dropwise to the vigorously stirred solution. After being maintained at −50° C. for 30 minutes, the solution was warmed up at room temperature and left to stirred overnight. An excess of ammonium hexafluorophosphate (0.025 g, 0.144 mmol) was added and stirred for 45 minutes. Then the product was extracted with dichloromethane and water (3×15 mL) and the combined organic phase was dried on MgSO₄. The targeted complex was then collected after filtration and removal of the solvent as an orange solid. Yield: 0.050 g (82%). M.P. 242-246° C. IR (ν/cm⁻¹): 1607 w (tetrazole C═N). ¹H NMR (δ/ppm, acetone-d⁶): 8.97 (d, 1H, H_{T4}, J=8.4 Hz), 8.66 (d, 1H, H_{T3}, J=8.5 Hz), 8.26 (d, 1H, H_{T6}, J=8.1 Hz), 8.19-8.07 (m, 4H, 4H_{A}), 7.99 (td, 1H, H_{T7}, J=8.2 Hz, J′=1.5 Hz), 7.94-7.83 (m, 4H, 2H_{A}, 2H_{B}), 7.71 (t, 1H, H_{A}, J=7.6 Hz), 7.35 (td, 1H, H_{A}, J=8.0 Hz, J′=1.6 Hz), 7.10-6.98 (m, 5H, 3H_{B}, H_{T8}, H_{T9}), 6.84 (td, 1H, H_{B}, J=7.5 Hz, J′=1.3 Hz), 6.43 (d, 1H, H_{B}, J=7.6 Hz), 6.17 (d, 1H, H_{B}, J=7.7 Hz), 4.59 (s, 3H, CH_{TMe}). ¹³C NMR (δ/ppm, acetone-d⁶): 168.8 (C_{A}), 168.6 (C_{A}), 168.1 (C_{T}), 152.0 (CH_{A}), 150.8 (C_{B}), 150.5 (CH_{A}), 148.9 (C_{B}), 147.5 (C_{T}), 145.2 (C_{B}), 144.7 (C_{B}), 143.2 (CH_{T}), 141.0 (C_{T}), 139.7 (CH_{A}), 139.6 (CH_{T}), 133.1 (CH_{B}), 132.5 (CH_{B}), 131.9 (CH_{B}), 131.6 (C_{T}), 131.3 (CH_{T}), 130.7 (CH_{A}), 130.4 (CH_{A}), 130.3 (CH_{A}), 129.0 (CH_{A}), 126.1 (CH_{B}), 125.4 (CH_{B}), 124.6 (CH_{T}), 124.1 (CH_{B}), 123.7 (CH_{B}), 123.6 (CH_{B}), 120.9 (CH_{T}), 120.8 (CH_{T}), 120.6 (CH_{A}), 42.6 (CH_{TME}). Anal. Calcd for [Ir(ppy)₂(MeTzQn)][PF₆]⋅0.17(CH₂Cl₂): C, 45.74; H, 2.93; N, 11.26. Found: C, 45.71; H, 2.60; N, 11.13.

[Ir(ppy)₂(MeTziQn)][PF₆]. [Ir(ppy)₂(TziQn)] (0.060 g, 0.086 mmol) was dissolved in (10 mL) and cooled down to −50° C. using an ethyl acetate/liquid nitrogen cool bath. Thereafter, a 0.1 M methyl trifluoromethanesulfonate solution in DCM (0.021 g, 0.129 mmol) was added dropwise to the vigorously stirred solution. After being maintained at −50° C. for 30 minutes, the solution was warmed up at room temperature and left to stirred overnight. An excess of ammonium hexafluorophosphate (0.028 g, 0.163 mmol) was added and stirred for 45 minutes. Then the product was extracted with dichloromethane and water (3×15 mL) and the combined organic phase was dried on MgSO₄. The targeted complex was then collected after filtration and removal of the solvent as an orange solid. Yield: 0.044 g (60%). M.P. 215-218° C. IR (ν/cm⁻¹): 1607 w (tetrazole C═N). ¹H NMR (δ/ppm, acetone-d⁶): 9.58 (d, 1H, H_{T9}, J=9.8 Hz), 8.27-8.20 (m, 4H, 4H_{A}), 8.15-8.10 (m, 2H, H_{T6}, H_{T7}), 8.05 (t, 2H, H_{A}, H_{T10}, J=5.7 Hz), 7.99-7.92 (m, 3H, 3H_{A}), 7.87 (d, 2H, H_{T4}, H_{T5}, J=7.7 Hz), 7.10-7.06 (m, 3H, 2H_{A}, H_{B}), 7.03-6.95 (m, 2H, 2H_{B}), 6.88 (td, 1H, H_{B}, J=7.6 Hz, J′=1.3 Hz), 6.35 (t, 2H, 2H_{B}, J=7.6 Hz), 4.69 (s, 3H, CH_{TME}). ¹³C NMR (6/ppm, acetone-d⁶): 168.8 (C_{A}), 168.5 (C_{A}), 168.2 (C_{T}), 151.2 (CH_{T}), 150.8 (CH_{T}), 150.1 (C_{B}), 145.9 (C_{T}), 145.5 (C_{B}), 145.2 (C_{B}), 145.1 (C_{B}), 143.0 (CH_{A}), 139.7 (CH_{A}), 139.7 (CH_{A}), 138.1 (C_{T}), 134.4 (CH_{T}), 132.8 (2CH_{B}), 132.2 (CH_{T}), 131.4 (CH_{B}), 130.6 (CH_{B}), 129.1 (CH_{A}), 128.8 (CH_{A}), 127.8 (C_{T}), 126.6 (CH_{T}), 125.9 (CH_{A}), 125.4 (CH_{T}), 124.5 (CH_{B}), 124.4 (CH_{B}), 123.8 (CH_{B}), 123.3 (CH_{B}), 120.7 (CH_{A}), 120.6 (CH_{A}), 42.7 (CH_{TME}). Anal. Calcd for [Ir(ppy)₂(MeTziQn)][PF₆]⋅0.4(CH₂Cl₂)⋅0.2(H₂O): C, 44.85; H, 2.95; N, 10.96. Found: C, 44.84; H, 2.90; N, 10.65.

X-Ray Structure Refinement Data

[Ir(F₂ppy)₂(TzPyCN)]. Crystallographic data for the structure were collected at 100(2) K on an Oxford Diffraction Gemini diffractometer fitted with Mo Kα radiation. Following analytical absorption corrections and solution by direct methods, the structure was refined against F² with full-matrix least-squares using the program SHELXL-2014 [ref: Sheldrick, G. M. (2008). Acta Cryst. A64, 112-122]. Empirical formula C_{30.50}H₁₈Cl₃F₄IrN₈; Formula weight 871.08; Temperature 100(2) K; Wavelength 0.71073 Å; Crystal system Monoclinic; Space group I2/a; Unit cell dimensions a=19.3193(4) Å, b=10.42280(10) Å, c=31.7649(6) Å, β=106.177(2)°; Volume 6142.96(19) Å³; Z 8; Density (calculated) 1.884 Mg/m³; μ 4.670 mm⁻¹; Crystal size 0.30×0.19×0.055 mm³; θ range for data collection 2.065 to 33.049°; Index ranges −25<=h<=28, −15<=k<=12, −47<=1<=46; Reflections collected 38758; Independent reflections 10992 [R(int)=0.0507]; Completeness to θ=31.00° 100.0%; Absorption correction Semi-empirical from equivalents; Max./min. transmission 1.000/0.873; Refinement method Full-matrix least-squares on F²; Data/restraints/parameters 10992/672/598; Goodness-of-fit on F² 1.038; Final R indices [I>2σ(I)] R1=0.0421, wR2=0.0857; R indices (all data) R1=0.0648, wR2=0.0948; Largest diff. peak and hole 2.125 and −1.357 e. Å⁻³. The benzonitrile group and one difluorophenyl ring are each disordered over two sets of sites with occupancy factors constrained to 0.5 after trial refinement. The solvent was modelled as three dichloromethane molecules with the occupancies of each constrained to 0.5 after trial refinement. All hydrogen atoms were added at calculated positions and refined by use of riding models with isotropic displacement parameters based on those of the parent atoms. Anisotropic displacement parameters were employed throughout for the non-hydrogen atoms.

[Ir(ppy)₂(MeTzPyMeCN)][PF₆]. Crystallographic data for the structure were collected at 200(2) K on an Oxford Diffraction Gemini diffractometer using Cu Kα radiation. Following analytical absorption corrections and solution by direct methods, the structure was refined against F² with full-matrix least-squares using the program SHELXL-2014 [ref: Sheldrick, G. M. (2015). Acta Cryst. C71, 3-8]. Empirical formula C_{37.38}H_{28.75}Cl_{2.75}F₆IrN₈P; Formula weight 1024.59; Temperature 100(2) K; Wavelength 1.54178 Å; Crystal system Monoclinic; Space group P2/n; Unit cell dimensions a=22.1244(4) Å, b=12.7352(2) Å, c=30.7626(4) Å, β=108.760(2)°; Volume 8207.2(2) Å³; Z 8; Density (calculated) 1.658 Mg/m³; M 8.889 mm⁻¹; Crystal size 0.250×0.151×0.029 mm³; θ range for data collection 2.968 to 67.3480; Index ranges −19<=h<=26, −15<=k<=15, −36<=1<=35; Reflections collected 51875; Independent reflections 14634 [R(int)=0.0509]; Completeness to 0=67.3480 99.2%; Absorption correction Analytical; Max. and min. transmission 0.731 and 0.195; Refinement method Full-matrix least-squares on F²; Data/restraints/parameters 14634/197/1057; Goodness-of-fit on F² 1.052; Final R indices [I>2σ(I)] R1=0.0541, wR2=0.1480; R indices (all data) R1=0.0717, wR2=0.1637; Largest diff. peak and hole 1.581 and −1.016 e·Å⁻³. The solvent was modelled as six dichloromethane molecules with their site occupancies constrained to 0.5 after trial refinement. Geometries of the solvent, anions and some phenyl rings were restrained to ideal values. All hydrogen atoms were added at calculated positions and refined by use of riding models with isotropic displacement parameters based on those of the parent atoms. Anisotropic displacement parameters were employed throughout for the non-hydrogen atoms.

Photophysical Measurements

Absorption spectra were recorded at room temperature using a Cary 4000 UV/Vis spectrometer. Uncorrected steady state emission and excitation spectra were recorded on an Edinburgh FLSP980-S2S2-stm spectrometer equipped with: i) a temperature-monitored cuvette holder; ii) 450 W Xenon arc lamp; iii) double excitation and emission monochromators; iv) a Peltier cooled Hamamatsu R928P photomultiplier tube (spectral range 200-870 nm). Emission and excitation spectra were corrected for source intensity (lamp and grating) and emission spectral response (detector and grating) by a calibration curve supplied with the instrument. According to the approach described by Demas and Crosby, luminescence quantum yields (Φₑₘ) were measured in optically dilute solutions (O.D.<0.1 at excitation wavelength) obtained from absorption spectra on a wavelength scale [nm] and compared to the reference emitter by the following equation:

$${Φ}_{x} = {Φ}_{r} ⌊ \frac{{A}_{r} ( {λ}_{r} )}{{A}_{x} ( {λ}_{x} )} ⌋ ⌊ \frac{{I}_{r} ( {λ}_{r} )}{{I}_{x} ( {λ}_{x} )} ⌋ [ \frac{{n}_{x}^{2}}{{n}_{r}^{2}} ] [ \frac{{D}_{x}}{{D}_{r}} ]$$

where A is the absorbance at the excitation wavelength (λ), I is the intensity of the excitation light at the excitation wavelength (λ), n is the refractive index of the solvent, D is the integrated intensity of the luminescence and Φ is the quantum yield. The subscripts r and x refer to the reference and the sample, respectively. The quantum yield determinations were performed at identical excitation wavelength for the sample and the reference, therefore cancelling the I(λᵣ)/I(λₓ) term in the equation. The quantum yields of complexes were measured against Rubipy in water (Φᵣ=0.028). Emission lifetimes (z) were determined with the time correlated single photon counting technique (TCSPC) with the same Edinburgh FLSP980-S2S2-stm spectrometer using either a pulsed picosecond LED (EPLED/EPL 377 nm, FHWM<800 ps) or a microsecond flashlamp. The goodness of fit was assessed by minimising the reduced χ² function and by visual inspection of the weighted residuals. The solvents used for the preparation of the solutions for the photophysical investigations were of LR grade and the water was deionised. Degassing of the dichlorometane solutions was performed using the freeze-pump-thaw method. Experimental uncertainties are estimated to be ±8% for lifetime determinations, ±20% for quantum yields, ±2 nm and ±5 nm for absorption and emission peaks, respectively.

TD-DFT Calculations

Time-dependent density functional theory calculations were performed with GAUSSIAN 09.33 prior to these calculations, the structures were relaxed at the CAM-B3LYP level of theory. The Ir atoms were treated with the Stuttgart-Dresden effective core potential, the Pople 6-311G** basis set was used for C, H, N, O, Cl, and S atoms, and the effect of the solvent was mimicked with the PCM solvation model, with parameters adequate for dichloromethane. The low-lying singlet-singlet excitation energies were calculated at the same level of theory, and the spectra were reproduced as the superposition of Gaussian functions with heights proportional to calculated intensities and a variance of 11 nm.

Cell Culture

H9c2 rat cardiomyoblast cell line was a kind gift from Professor Janna Morrison (University of South Australia). The H9c2 cells were maintained in high-glucose (4500 mg/L) DMEM medium (Sigma-Aldrich, USA) containing 10% fetal bovine serum (FBS; In Vitro Technologies, USA) and 2 mM L-glutamine (Sigma-Aldrich, USA) at 37° C. and 5% CO₂. The H9c2 cells were cultured in 75 mm² flasks, and cells (1×10⁶ cells/mL) that had been passaged, from two up to five times were used for experiments. For live cell imaging and MTS assay, the H9c2 cells (1×10⁵ cells/mL) were cultured overnight in either ibidi μ-Slide 8 wells in a final volume of 250 μL or 96-well microtiter plate in a final volume of 200 μL in absence of Ir-based molecular probes.

Confocal Microscopy

The H9c2 cells were incubated with 20 μM of Ir-based molecular probes for 30 minutes in FBS-free media (300 μL) at 37° C. and 5% CO₂. These cells were washed twice with phosphate buffered saline (PBS) and once with FBS-free media, and then co-stained with either ER-Tracker™ Red (BODIPY® TR Glibenclamide), LysoTracker® Red DND-99, MitoTracker® Red CMXRos (1H,5H,11H,15H-Xantheno[2,3,4-ij:5,6,7-i′j′]diquinolizin-18-ium, 9-[4-(chloromethyl)phenyl]-2,3,6,7,12,13,16,17-octahydro-, chloride), β-BODIPY® FL C₅-HPC (2-(4,4-Difluoro-5,7-Dimethyl-4-Bora-3a,4a-Diaza-s-Indacene-3-Pentanoyl)-1-Hexadecanoyl-sn-Glycero-3-Phosphocholine) or BODIPY® 500/510 C₁, C₁₂ (4,4-Difluoro-5-Methyl-4-Bora-3a,4a-Diaza-s-Indacene-3-Dodecanoic Acid), which were obtained from Life Technologies Australia Pty Ltd, Australia. Cell biology analysis was carried out with a minimum of two experimental groups. Imaging analysis was performed using a Nikon A1+ confocal microscope equipped with 403 nm, 488 nm, 561 nm and 640 nm solid-state lasers (Nikon, Japan) and OKOLab Microscope Incubator (Okolab USA Inc., USA). The final preparation of the images was conducted with Adobe Photoshop CC (Adobe Systems Inc., USA).

Two-Photon Microscopy

The H9c2 cells were incubated with 20 μM of Ir-based molecular probes for 30 minutes in FBS-free media (300 μL) at 37° C. and 5% CO₂. These cells then were washed with phosphate buffered saline (PBS) and imaged using Zeiss LSM710 META NLO inverted microscope supplemented with a two-photon Mai-Tai®, tunable Ti:Sapphire femtosecond pulse laser (710-920 nm, Spectra-Physics). Fluorescence of Ir-based molecular probes was detected using the following settings: two-photon excitation wavelength 830 nm, beam splitter MBS 690+, emission interval 520-650 nm. The H9c2 cells were exposed to two-photon illumination between 810-840 nm. A continuum of lambda stack images was captured across the emission spectrum 416-728 nm. The images were acquired using a Plan-APOCHROMAT 63X/NA1.4 oil immersion objective. The final preparation of the images was conducted with Adobe Photoshop CC (Adobe Systems Inc., USA).

MTS Cell Viability Assay

Cellular NADPH-dependent redox activity was measured using CellTiter 96® AQueous Non-Radioactive Cell Proliferation Assay (MTS) according to the manufacturer's instruction (Promega, USA). The H9c2 cells were incubated with Ir-based molecular probes for 24 hours in FBS-free media (200 μL). The control cells were incubated for the same length of time in either FBS-free media or 0.2% DMSO. Then 20 μL of MTS reagents was added into each well and the cells were incubated for 2 hours at 37° C. and 5% CO₂. The absorbance of formazan dye produced by viable cells was measured by EnVision multi-label plate reader (PerkinElmer, Beaconsfield, UK) at 490 nm. Data represents the mean±SEM of three biological replicates for each group.

### REFERENCES

1 S. Neumann, D. Dihler, D. Strihl and W. H. Binder, Polym. Chem., 2016, 7, 2342-2351.
2 N. G. R. Hearns, R. Clérac, M. Jennings and K. E. Preuss, Dalton Trans., 2009, 3193-3203.
3 A. Wada, Q. Zhang, T. Yasuda, I. Takasu, S. Enomoto and C. Adachi, Chem. Commun., 2012, 48, 5340-2.
4 V. Fiorini, A. M. Ranieri, S. Muzzioli, K. D. M. Magee, S. Zacchini, N. Akabar, A. Stefan, M. I. Ogden, M. Massi and S. Stagni, Dalt. Trans., 2015, 44, 20597-20608.
5 N. M. La, R. Burgard and C. Wentrup, 2004, 2033-2036.
6 S. Sprouse, K. a. King, P. J. Spellane and R. J. Watts, J. Am. Chem. Soc., 1984, 106, 6647-6653.
7 M. Lepeltier, F. Dumur, J. Marrot, E. Contal, D. Bertin, D. Gigmes and C. R. Mayer, Dalt. Trans., 2013, 42, 4479-86.
8 S. Stagni, S. Colella, A. Palazzi, G. Valenti, S. Zacchini, F. Paolucci, M. Marcaccio, R. Q. Albuquerque and L. De Cola, Inorg. Chem., 2008, 47, 10509-10521.

### Example 2—Staining of Cancer Cells

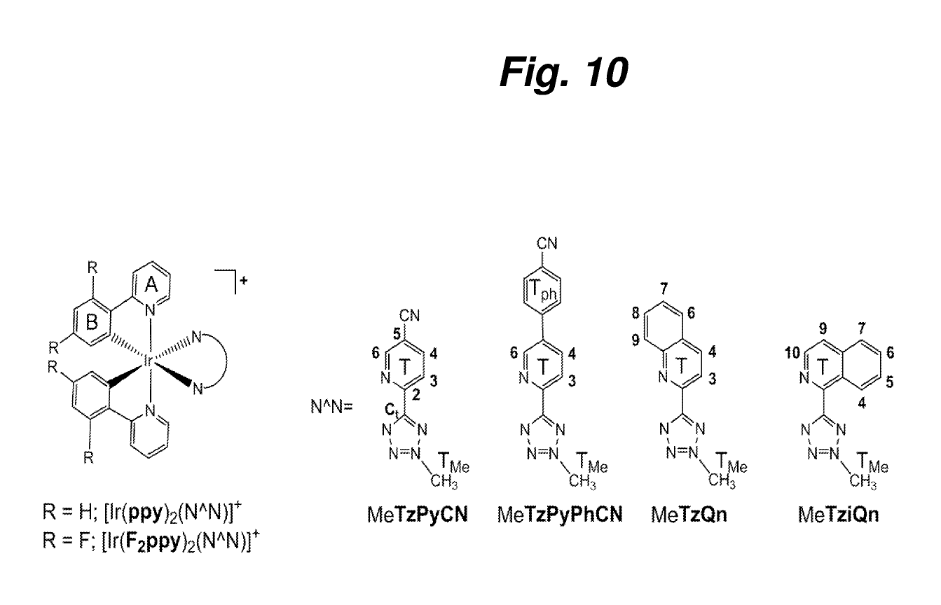

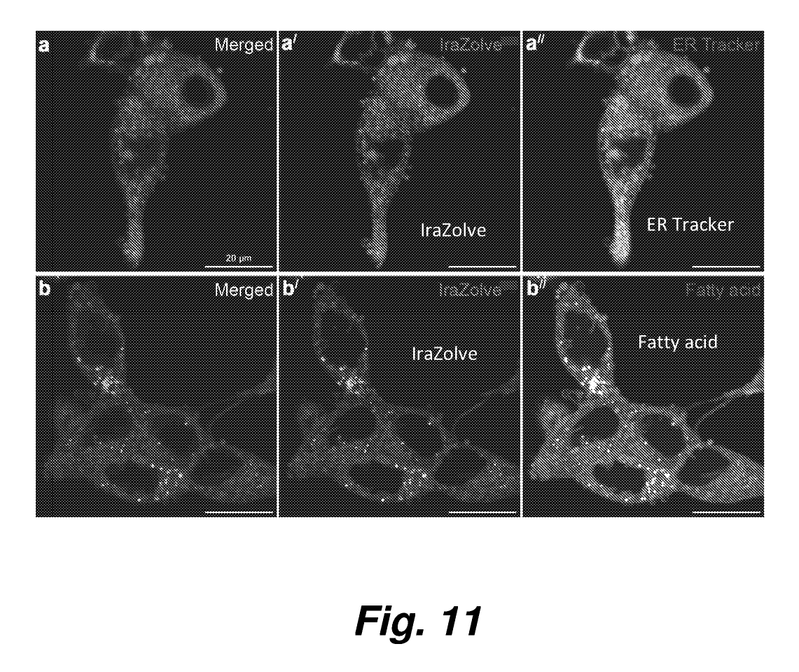

We took three key fluorescent dyes marketed as lipid stains, Filipin, BODIPY® 493/503 and ReZolve-L1™ (Rezolve Scientific, Australia) and the fluorescent dye [Ir(ppy)₂(TzPyCN)] (also referred to herein as “IraZolve”) to see if we can differentiate under the microscope non-malignant control PNT1a cells from prostate cancer cell lines based on lipid staining. Filipin is a stain for free cholesterol and it was of interest to investigate the ability of Filipin to detect cholesterol differences in prostate cell lines. BODIPY® 493/503 (4,4-difluoro-4-bora-3a,4a-diaza-s-indacene) is a neutral lipid stain, which has been shown to detect triacylglycerides and cholesteryl esters in lipid droplets. Up-regulated levels of cholesteryl esters were shown in the FTIR to be a characteristic of prostate cancer cells (data not shown). ReZolve-L1™ has been marketed as a fluorescent dye which localises in areas of high polar lipid content in mammalian adipocytes and Drosophila fat body tissues. As described herein, IraZolve [Ir(ppy)₂(TzPyCN)] is a fluorescent dye that stains the endoplasmic reticulum, where both phospholipids and triacylglycerides are synthesised, and it also co-localises with fatty acids in lipid droplets (FIG. 11).

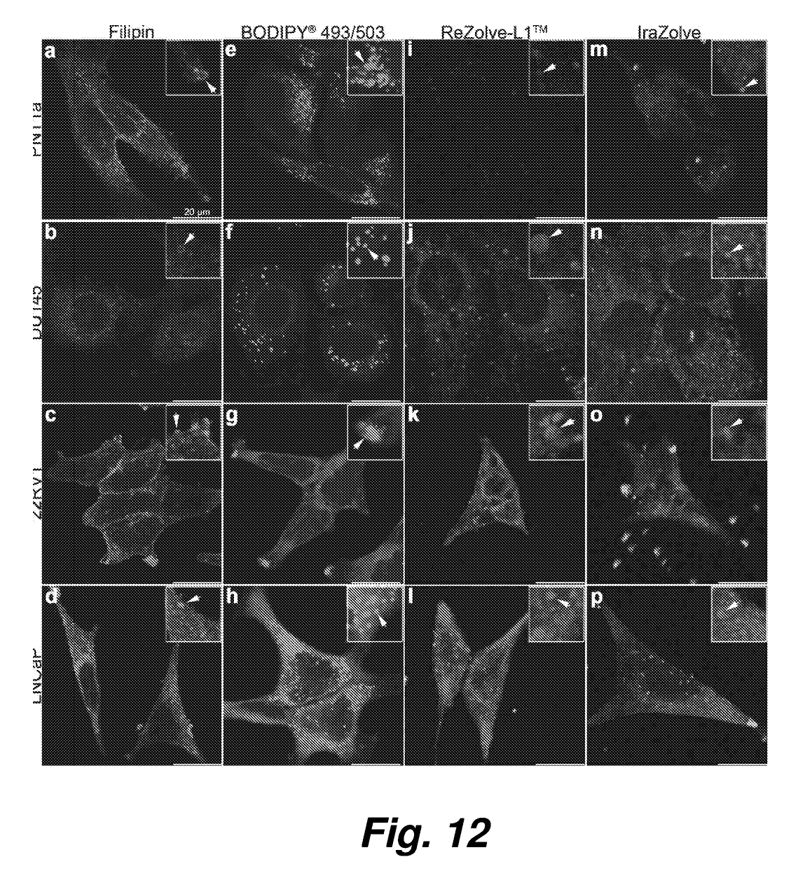

The fluorescent dyes, Filipin and BODIPY® 493/503, were incubated for 30 minutes with each of the four prostate cell lines (i.e. PNT1a, DU145, 22RV1 and LNCaP), fixed in 4% paraformaldehyde and imaged under confocal microscopy, which was supplemented with two-photon Mai-Tai® and Argon lasers. Prostate cell lines grown on coverslips were stained with ReZolve-L1™ and IraZolve [Ir(ppy)₂(TzPyCN)] in serum-free media for 30 minutes at 37° C. and 5% CO₂ and imaged in live mode, avoiding the process of fixation. From this experiment, different staining patterns between prostate cell lines and the fluorescent dyes were clearly seen (FIG. 12). For example, the level of diffuse cytosolic staining vs membrane staining with Filipin varied between cell lines (FIG. 12 a-d). In non-malignant control PNT1a and prostate cancer LNCaP cells (FIG. 12a, d), a number of small vesicles were brightly stained with Filipin. Prostate cancer 22RV1 cells had quite bright vesicular and plasma membrane staining in comparison to DU145 (FIG. 12b, c), which had a relatively low level. This led us to consider that Filipin did not depict differences in the levels of free cholesterol and gangliosides in prostate cells as this fluorescent dye poorly stained DU145 cells, which contained high concentrations of these two lipids. Differences in the 22RV1 and LNCaP staining pattern in comparison to PNT1a and DU145 were apparent with BODIPY® 493/503 fluorescent dye. DU145 cells stained with BODIPY® 493/503 showed localised staining to lipid droplets (FIG. 12f) similar to that observed in PNT1a cells (FIG. 12e). In 22RV1 cells (FIG. 12g), BODIPY® 493/503 showed a diffused cytosolic staining and localised staining to lipid droplets; remarkably, lipid droplets accumulated in the cellular projections. The fluorescence of ReZolve-L1™ was low in non-malignant control PNT1a cells, when compared to prostate cancer cells (FIG. 12i-l). A very similar staining pattern to BODIPY® 493/503 was observed for ReZolve-L1™ when DU145 cells were compared with LNCaP (FIG. 12j, 12l). The only difference being that the staining of DU145 cells with ReZolve-L1™ was less localised with the lipid droplets in comparison to BODIPY® 493/503 stain. 22RV1 cells showed a diffused staining with ReZolve-L1™ (FIG. 12k). These positive results prompted us to analyse staining of the prostate cells with the fluorescent dye IraZolve (FIG. 12m-p). In prostate cancer cells (i. e. DU145, 22RV1 and LNCaP), the pattern of staining with IraZolve [Ir(ppy)₂(TzPyCN)] was almost identical to that observed for ReZolve-L1™. In contrast to ReZolve-L1™ (FIG. 12i), PNT1a cells showed a diffused staining with IraZolve [Ir(ppy)₂(TzPyCN)] in cytosol, with some accumulation in lipid droplets (FIG. 12m). These data showed that the fluorescent dyes investigated have the ability to show differences between cell lines studied. However, the most striking difference in FIG. 12 was with ReZolve-L1™, which was barely detectable in non-malignant control PNT1a cells; slight staining was observed in small intracellular vesicles (FIG. 12i). In contrast, the intensity of staining in the malignant cell lines DU145, 22RV1 and LNCaP with ReZolve-L1™ was significant. The highest intensity staining as stated was with LNCaP (FIG. 12l). Moreover, it has been noted that BODIPY® 493/503 and ReZolve-L1™ had different pattern of staining, suggesting that ReZolve-L1™ could depict the differences in these polar lipid content in prostate cell lines. Therefore, staining of prostate cells with ReZolve-L1™ resulted in the differentiation between non-malignant control PNT1a and prostate cancer DU145, 22RV1 and LNCaP cell lines. The difference in ReZolve-L1™ staining observed between 22RV1 and LNCaP, in comparison to DU145 and PNT1a cells, may be accounted for by the aggressive nature of the cell types. Consequently, this difference in staining pattern may also be useful for the differentiation of cancer cell line as uncovering a fluorescent dye or a combination of fluorescent dyes, which could identify aggressive types of prostate cancer, and this has significance in the choice of disease treatment.

The IraZolve [Ir(ppy)₂(TzPyCN)] staining was observed in the endoplasmic reticulum and small intracellular vesicles in prostate cell lines. The intensity of staining in the malignant cell lines DU145, 22RV1 and LNCaP with IraZolve [Ir(ppy)₂(TzPyCN)] was significantly greater, when compared to non-malignant control PNT1a. The staining in PNT1a cells was barely detectable.

Cell Lines and Culture Conditions.

Human prostate non-malignant control PNT1a and prostate cancer 22RV1, LNCaP (clone FCG) and DU145 cell lines were obtained from the European Collection of Cell Cultures via CellBank Australia (Children's Medical Research Institute, Westmead, NSW, Australia). PNT1a and 22RV1 cells were maintained in RPMI-1640 medium (#R0883, Sigma-Aldrich, USA) supplemented with 10% fetal bovine serum (#IVT3008403, In Vitro Technologies, Australia) and 2 mM L-glutamine (#25030-081, Gibco®, USA). For LNCaP, this culture medium was also supplemented with 10 mM HEPES (#H0887, Sigma-Aldrich, USA) and 1 mM sodium pyruvate (#S8636, Sigma-Aldrich, USA). DU145 cells were cultured in MEM medium (#M5650, Sigma-Aldrich, USA), supplemented with 10% fetal bovine serum, 2 mM L-glutamine and 1 mM sodium pyruvate. The prostate cell lines were incubated at 37° C. with 5% CO₂ in a Sanyo MCO-17AI humidified incubator (Sanyo Electric Biomedical Co., Ltd.). Prior to experiments, cells reached approximately 80% confluence were washed with sterile phosphate buffered saline (PBS; #D8537, Sigma-Aldrich, USA) and detached using TrypLE™ Express (#12604-021, Gibco®, USA).

Fixing and Staining Cells with Lipid Dyes.

Non-malignant control PNT1a and prostate cancer DU145, 22RV1 and LNCaP cell lines were cultured on coverslips in six well plates containing 3 mL of culture media. The cells were quickly rinsed with sterile PBS, fixed with 4% paraformaldehyde for 30 minutes at room temperature and then washed with sterile PBS for 30 minutes (3×10 minutes). Non-malignant control PNT1a and three prostate cancer cell lines were stained with BODIPY® 493/503 (1:100, #D3922, Life Technologies, USA) and Filipin complex from Streptomyces filipinensis (1:1000, #F9765, Sigma-Aldrich, USA) for 30 minutes. The cells were briefly washed with sterile PBS for 30 minutes at room temperature and mounted for imaging.

Staining of Live Cells with ReZolve-L1™ and IraZolve [Ir(Ppy)₂(TzPyCN)]

The four prostate cell lines grown on coverslips were rinsed with sterile PBS and incubated with 20 μM of either ReZolve-L1™ (Rezolve Scientific, Australia) or IraZolve [Ir(ppy)₂(TzPyCN)] or in serum-free media for 30 minutes at 37° C. and 5% CO₂. Following staining, the cells were washed with sterile PBS and mounted for imaging.

Confocal and Two-Photon Microscopy.

Prostate cells stained with lipid dyes were imaged with a Ziess LSM710 META NLO inverted microscope (Zeiss, Germany), which was supplemented with a two-photon Mai-Tai®, tuneable Ti:Sapphire femtosecond pulse laser (710-920 nm, Spectra-Physics, USA). All imaging experiments were carried out at room temperature. Imaging BODIPY® 493/503 was performed using Argon-gas solid-state laser (Zeiss, Germany). Filipin was detected using two-photon excitation wavelength 720 nm, beam splitter MBS 690+ and emission interval 407-480 nm. ReZolve-L1™ fluorescence was acquired at two-photon excitation wavelength 820 nm, beam splitter MBS 690+ and emission interval 493-601 nm. IraZolve [Ir(ppy)₂(TzPyCN)] fluorescence was acquired using wo-photon excitation wavelength 830 nm, beam splitter MBS 690+ and emission interval 550-658 nm. All images were acquired using a Plan-APOCHROMAT 63X/NA1.4 oil immersion objective. Each confocal micrograph represented 1.0 μm thin optical sections. Nikon microscope->IraZolve [Ir(ppy)₂(TzPyCN)] co-staining with ER Tracker and Fatty acids).

Image Processing.

Representative images and graphs were collated using Adobe Photoshop CC (Adobe Systems Inc, USA).

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of intracellular imaging of a cell, the method comprising exposing the cell to a complex comprising a phenylpyridine iridium (III) (and/or a functional derivative thereof) and a tetrazolato compound and imaging the complex in the cell.

2. The method according to claim 1, wherein the tetrazolato compound comprises a nitrogen containing aromatic heterocyclic group.

3. The method according to claim 2, wherein the nitrogen containing aromatic heterocyclic group comprises a pyridyl or a pyrazinyl group and/or a functional derivative thereof.

4. The method according to claim 1, wherein the complex comprises one or more of the following complexes: 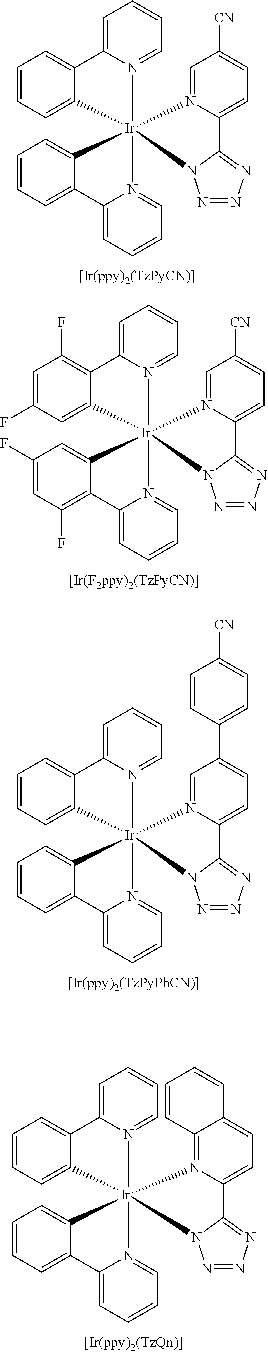 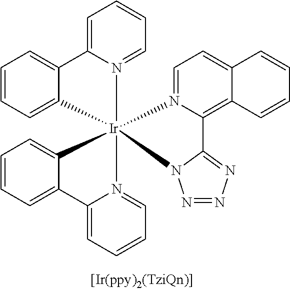 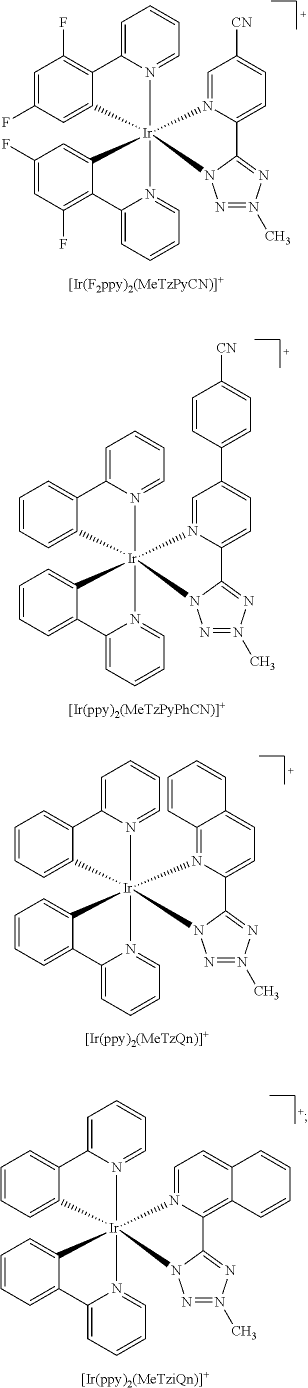
and/or a salt, a derivative, and/or an isomer of any of the aforementioned complexes.

5. The method according to claim 1, wherein the complex is a neutral complex.

6. The method according to claim 5, wherein the complex localises to one or more of endoplasmic reticulum, lipid droplets and acidic vesicles in the cell.

7. The method according to claim 1, wherein the complex is a cationic complex.

8. The method according to claim 7, wherein the complex localises to mitochondria in the cell.

9. The method according to claim 1, wherein the cell is a live cell.

10. (canceled)

11. The method according to claim 1, wherein the cell is a fixed cell.

12. The method according to claim 1, wherein the cell is present in vivo, in a cell sample, a sample of live cells, a cell extract, a biopsy, a bodily fluid sample, a blood sample, a urine sample, a saliva sample, a biological sample and/or an extract, component, derivative, processed form or purified form of any of the aforementioned.

13. (canceled)

14. (canceled)

15. The method according to claim 1, wherein the method is used for imaging of a live cell, in vivo imaging, to label a cell, to detect or label a cellular structure, to detect or label endoplasmic reticulum, to detect or label lipid droplets, to detect or label acidic vesicles, to detect or label mitochondria, to detect or label a vesicular compartment, to detect or label a non-cancerous cell and/or a cancerous cell, to identify a non-cancerous cell or a cancerous cell, to screen for cancerous cells, and to distinguish a cancerous cell from a non-cancerous cell.

16.-21. (canceled)

22. A kit for performing a method according to claim 1.

23.-25. (canceled)

26. An intracellular imaging agent, the agent comprising a complex comprising a phenylpyridine iridium (III) (and/or a functional derivative thereof) and a tetrazolato compound.

27. (canceled)

28. (canceled)

29. A method for labelling a cell, the method comprising using an agent according to claim 26 to label the cell.

30. (canceled)

31. A method of identifying a complex for intracellular imaging or labelling of a cell, the method comprising:
providing a candidate complex comprising a complex comprising a phenylpyridine iridium (III) (and/or a functional derivative thereof) and a tetrazolato compound;
determining the ability of the candidate complex to intracellularly image or label a cell; and
identifying the candidate complex as a complex for intracellular imaging or labelling of a cell.

32. The intracellular imaging agent according to claim 26, wherein the tetrazolato compound comprises a nitrogen containing aromatic heterocyclic group.

33. The intracellular imaging agent according to claim 26, wherein the nitrogen containing aromatic heterocyclic group comprises a pyridyl or a pyrazinyl group and/or a functional derivative thereof.

34. The intracellular imaging agent according to claim 26, wherein the complex comprises one or more of the following complexes: 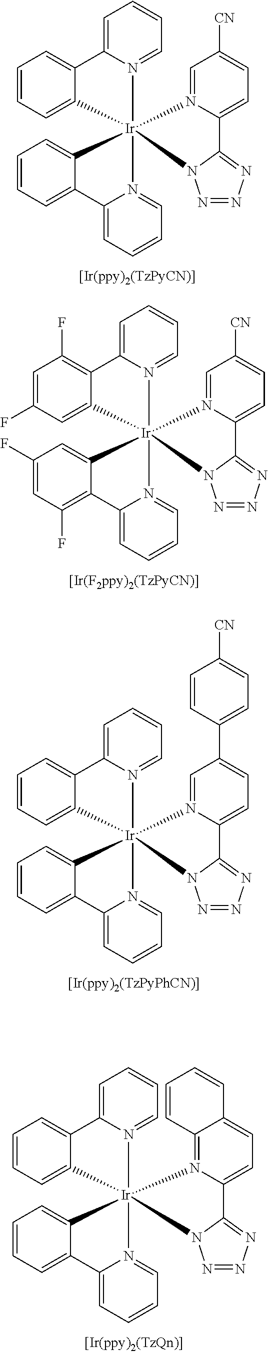 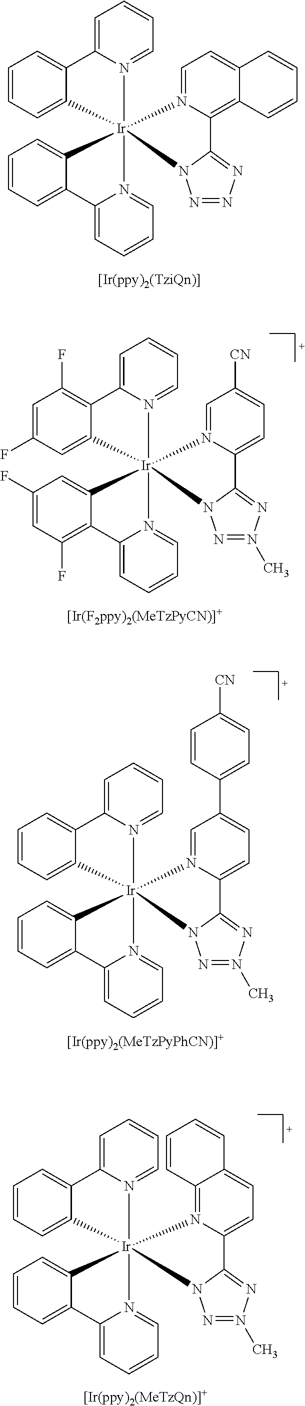 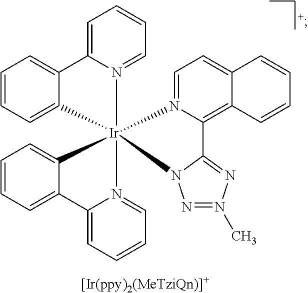
and/or a salt, a derivative, and/or an isomer of any of the aforementioned complexes.

35. A kit for intracellular imaging of cells, the kit comprising an agent according to claim 26.
